(19) United States
(12) Patent Application Publication
(10) Pub. No.: US 2024/0424217 A1
(43) Pub. Date: Dec. 26, 2024

(54) ELECTRICAL POWER SUPPLY DEVICE FOR AN ELECTRONIC UNIT OF A DRUG DELIVERY DEVICE

(71) Applicant: Sanofi, Paris (FR)

(72) Inventors: Stefan Alt, Frankfurt am Main (DE); Tim Glasser, Rüsselsheim (DE); Michael Helmer, Frankfurt am Main (DE); Stephan Mücke, Rüsselsheim (DE); Peter Nober, Rüsselsheim (DE); Michael Schabbach, Frankfurt am Main (DE); Martin Vitt, Frankfurt am Main (DE)

(21) Appl. No.: 18/705,911

(22) PCT Filed: Nov. 1, 2022
(86) PCT No.: PCT/EP2022/080404

§ 371 Date: Apr. 29, 2024

(30) Foreign Application Priority Data
Nov. 3, 2021 (EP) 21315219.2

(51) Int. Cl. A61M 5/24; A61M 5/315

(52) U.S. Cl. CPC A61M 5/24; A61M 2005/31588; A61M 2205/8206

(57) ABSTRACT

The present disclosure relates to an electrical power supply device for an electronic unit of a drug delivery device. The electrical power supply device includes: a body extending along a longitudinal direction and comprising a distal end and a proximal end, a rechargeable energy reservoir arranged inside or on the body, a supply interface electrically connected to the rechargeable energy reservoir, the supply interface being configured to electrically couple with a electrical interface of the drug delivery device, wherein the electrical interface is electrically connected to the electronic unit and wherein the supply interface is operable to transfer electric energy from the rechargeable energy reservoir via the electrical interface to the electronic unit, and a charging interface electrically connected to the rechargeable energy reservoir and operable to connect to an external power supply for charging the rechargeable energy reservoir.

### CROSS REFERENCE TO RELATED APPLICATIONS

The present application is the national stage entry of International Patent Application No. PCT/EP2022/080404, filed on Nov. 1, 2022, and claims priority to Application No. EP 21315219.2, filed on Nov. 3, 2021, the disclosures of which are incorporated herein by reference.

### TECHNICAL FIELD

The present disclosure relates to an electric power supply device for an electronic unit of a drug delivery device. In another aspect the present disclosure relates to a drug delivery device a whip with such an electric power supply device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, e.g. injection devices and needle based injection system (NIS) devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Needle based injection to be conducted with a variety of injection devices is more and more associated with measuring and logging data regarding the amount of medicament set and set or dispensed or injected at a particular date or time. For this, injection devices may be provided with an electronic circuit providing data acquisition and/or data logging as well as communication of collected or measured dispensing related data.

Electronic circuits or electronic units may be provided by a supplementary device or an add-on device configured for releasable attachment to the injection device and being operable to log injection-related data during use of the injection device. Such electronic circuits or electronic units may be also implemented or integrated into the injection device itself. Moreover, electronic circuits for data logging may be also provided with a communication interface so as to transmit previously stored injection-related data to an external electronic device for further data processing.

Since an electronic circuit consumes electrical power such electronic units are typically equipped with an electrical power supply, such as a battery. The battery of such electronic circuit requires a respective construction space, which makes it difficult to further miniaturize such electronic units. Since construction space is rather limited in hand held or portable drug delivery devices it is a particular aim to further miniaturize a battery and/or an electronic unit integrated or attached to a drug delivery device.

Generally, the electrical power supply of the electronic unit may comprise a rechargeable battery. But also recharging of such batteries not being directly accessible from outside a drug delivery device might be quite challenging.

### SUMMARY

An aim of the present disclosure is to provide improvement with regard to electric energy supply for electronic units of devices such as portable drug delivery devices. Moreover, the subject matter of the present disclosure allows for patient safety as well as operability of the drug delivery device to be improved and simplified for a user.

In one aspect, the present disclosure relates to an electrical power supply device for an electronic unit of a drug delivery device. The electrical power supply device comprises a body extending along a longitudinal direction and comprising a distal end and a proximal end. The distal end and a proximal end are opposite ends or end sections of the body and hence of the electrical power supply device. The electrical power supply device further comprises a rechargeable energy reservoir arranged inside or on the body. The electrical power supply device further comprises a supply interface electrically connected to the rechargeable energy reservoir. The supply interface is configured to electrically couple with an electrical interface of the drug delivery device.

The electrical interface of the drug delivery device is electrically connected to the electronic unit of the drug delivery device. Here, the supply interface of the electrical power supply device is operable or configured to transfer electric energy from the rechargeable energy reservoir via the electrical interface to the electronic unit of the drug delivery device. The electrical power supply device further comprises a charging interface electrically connected to the rechargeable energy reservoir. The charging interface is operable to connect to an external power supply for charging the rechargeable energy reservoir.

Hence, the presently disclosed electrical power supply device comprises a rechargeable energy reservoir, which primarily serves to charge and/or to provide electrical energy to an electronic unit of a drug delivery device. Typically, the rechargeable energy reservoir is operable to charge a local electric energy reservoir, e.g. a battery, of the electronic unit. Hence, providing of electrical energy to the drug delivery device by the supply interface may come along with a charging of a local battery of the electronic unit or of the drug delivery device. In this way the rechargeable energy reservoir of the electrical power supply device acts and behaves as a charger for a battery of the electronic unit of the drug delivery device.

The rechargeable energy reservoir of the electrical power supply device itself is also rechargeable. In this way and since the electrical power supply device is equipped with a rechargeable energy reservoir, e.g. for charging a battery of the electronic unit of the drug delivery device a storage capacity of the rechargeable energy reservoir can be reduced, thus allowing to further reduce the geometric dimensions of the rechargeable energy reservoir and of the electrical power supply device as such. This provides the benefit to miniaturize the electrical power supply device and to provide an elegant and user-attractive solution for practically implementing the electrical power supply device.

With some examples, the electrical power supply device may be integrated into a component of the drug delivery device. Reducing of the storage capacity of the rechargeable energy reservoir allows for a respective compact design of the electrical power supply device. This enables a smooth integration of the electrical power supply device, e.g. in a detachable component of the drug delivery device.

With another example, the electrical power supply device provides a kind of a power bank for providing electric energy to the electronic unit of the drug delivery device on demand. The electrical power supply device is particularly and selectively usable or activatable when a local battery onboard of the electronic unit should be depleted. Then and by establishing an electric coupling or electrical connection between the supply interface of the electrical power supply device and the electronic unit of the drug delivery device the local battery of the electronic unit, which is of rechargeable type, can be charged.

If such a charging procedure is conducted numerous times the rechargeable energy reservoir of the electrical power supply device may be also depleted. Here, the charging interface of the electrical power supply device may then be used to connect the electrical power supply device with an external power supply for charging the rechargeable energy reservoir.

According to another example, the charging interface comprises a power connector. The power connector may be configured to establish or to contribute to a galvanic connection with a complementary shaped power connector of the external power supply. The power connector of the charging interface may be implemented in accordance to a well-established power connector standard. It may comprise a USB connector or any other standardized power connector.

With some examples the charging interface and hence the power connector comprises one of a plug and a socket to detachably connect to a complementary shaped counter socket or counter plug of the external power supply. In this way, there can be provided a rather easy and straightforward approach of how to charge or recharge the rechargeable energy reservoir of the electrical power supply.

According to a further example, the charging interface comprises an inductive coupler configured to couple inductively with a complementary-shaped counter inductive coupler of the external power supply. Here, charging of the rechargeable energy reservoir may be conducted or executed on the basis of a conductive, hence non-contact electrical coupling between the inductive coupler of the electrical power supply device and the complementary or corresponding counter inductive coupler of the external power supply.

An inductive coupler enables a wireless charging and may be provided instead of one of a plug and a socket of the charging interface. With an inductive coupler, the charging interface of the electrical power supply device may be void of a socket or plug. A wireless charging interface of the electrical power supply device may enable and support a rather attractive design of the electrical power supply device being void of a plug or a socket of a power connector.

According to a further example, the rechargeable energy reservoir is arranged inside the body. The supply interface is arranged at the proximal end or extends into the proximal end of the body. The charging interface is arranged at the distal end. Arranging the supply interface and the charging interface at opposite longitudinal ends of the rechargeable energy reservoir is beneficial in many aspects. For example, the supply interface of the electrical power supply device may be electrically coupled or may remain electrically coupled with the electrical interface of the drug delivery device while at the same time the charging interface may be electrically coupled to the external power supply. This way, there may be provided a rather direct charging or recharging of a local battery of the electronic unit, wherein the rechargeable energy reservoir only acts as a buffer for electrical energy.

Moreover, by having the supply interface and the charging interface at opposite ends of the body the respective interfaces do not spatially overlap, which allows to connect the supply interface with the electronic unit independently from a connection between the charging interface and the external power supply. In this way, the recharging process of the rechargeable energy reservoir can decoupled from a charging process of the local battery of the electronic unit, which is supplied by the rechargeable energy reservoir. In this way, the timescales for the respective charging processes, namely for a first charging process by way of which the electronic unit is charged or supplied with electrical energy from the rechargeable energy reservoir and a second charging process, during which the rechargeable energy reservoir itself is provided with electrical energy from the external electric power supply via the charging interface, can be implemented almost totally independent from each other. This way, the flexibility of using the electrical power supply device can be enhanced and user acceptance of such an electrical power supply device can be increased.

According to a further example, the body of the electrical power supply device comprises a mechanical counter interface at the proximal end to detachably fasten the body to a complementary shaped mechanical interface of the drug delivery device. A mechanical fastening of the body of the electrical power supply device to the mechanical counter interface, e.g. provided on or in a housing of the drug delivery device is of practical use to supply electrical energy to the electronic unit of the drug delivery device.

A mutual fastening of the body of the electrical power supply device with the drug delivery device may provide a durable electrical connection or electrical coupling between the supply interface of the electrical power supply device and the electrical interface of the drug delivery device.

According to a further example, the mechanical counter interface of the body of the electrical power supply device comprises a counter fastener to detachably engage with a complementary shaped fastener that is provided at the mechanical interface of the drug delivery device. With some examples the fastener and the counter fastener form one of a snap fit connection or a clip joint, a bayonet joint or a screw threaded joint. By way of the counter fastener engaging with the fastener of the drug delivery device the electrical power supply device, at least the body thereof, can be kept in a stationary charging position relative to the drug delivery device.

Typically, the electrical power supply device is a portable device. The charging interface may be electrically coupled to the external power supply independent of the respective configuration of the supply interface. The charging interface may be coupled to the external power supply while the supply interface is disconnected from the electrical interface of the drug delivery device.

With other examples, the charging interface can be coupled to the external power supply while the supply interface is connected with the electrical interface of the drug delivery device.

According to a further example, the body of the electrical power supply device comprises a side wall confining a receptacle. The receptacle may be open towards a proximal direction and may be shaped and sized to receive at least a portion of the drug delivery device. With some examples the electrical power supply device may be integrated into a housing or into a housing portion of the drug delivery device. The receptacle may be sized and shaped to receive a portion, e.g. a distal portion of the drug delivery device. When the drug delivery device is implemented as a handheld injection device, such as an injection pen, the receptacle of the body of the electrical power supply device may be sized and configured to receive a cartridge or container holder portion of the distal end of the housing of the drug delivery device.

Hence, the electrical power supply device may be integrated into a protective cap of a pen-type drug delivery device. With further examples, the electrical power supply device may comprise a protective cap or may be implemented as a protective cap of a handheld injection device, such as an injection pen.

According to a further example, the mechanical counter interface of the body of the electrical power supply device is integrated into or is provided at a proximal end of the sidewall of the body. With some examples the mechanical counter interface may be provided at a proximal open end of the receptacle of the body. In this way, there can be provided a rather easy and straightforward mutual mechanical detachable connection between the body of the electrical power supply device and a housing or a housing component of the drug delivery device.

According to a further example, the supply interface of the electrical power supply device comprises a first electric supply conductor path electrically connecting the supply interface with the rechargeable energy reservoir. The supply interface further comprises a second electric supply conductor path isolated from the first electric supply conductor path and electrically connecting the supply interface with the rechargeable energy reservoir. The first and/or the second electric supply conductor paths may extend along the longitudinal direction. They may extend from the rechargeable energy reservoir towards the proximal end of the body. When the rechargeable energy reservoir is located at or near the distal end of the body the first and the second electric supply conductor paths may extend almost all along the longitudinal extend of the body of the electrical power supply device.

With some examples, the body of the electrical power supply device may comprise a somewhat tubular or cylindrical shape or structure. Here, the first and the second electric supply conductor paths may be separated from each other along a circumferential or tangential direction of the sidewall of the body. The first and/or the second electric supply conductor paths may comprise a somewhat longitudinal straight shape. In this way, material and expenditure required for implementing the first and/or the second electric supply conductors can be reduced to a minimum thereby still providing an electric coupling or electrical connection between the rechargeable energy reservoir, e.g. located at or near the distal end of the body, and the proximal end of the body, which may be not only mechanically but also electrically connected to the electrical power supply device.

According to a further example, at least one of the first electric supply conductor path and the second electric supply conductor path is arranged on an inside surface or on an outside surface of the sidewall of the body of the electrical power supply device.

With some examples, one of the first electric supply conductor path and the second electric supply conductor path is arranged on the inside surface of the sidewall and the other one of the first electric supply conductor path and the second electric supply conductor path is provided on the outside surface of the sidewall. In this way and when the material of the sidewall is electrically isolating the first and the second electric supply conductor paths are inherently electrically isolated by the sidewall of the body. This may equally apply to configurations, in which the first electric supply conductor path and the second supply conductor path are located at a spatial distance from each other on the same or on a common side of the sidewall.

With some examples, at least one of the first and the second electric supply conductor paths comprises a metallized structure or metallic structure provided on or inside the body. Typically, a portion of the body, e.g. its sidewall or a sidewall portion may be coated, printed, laminated or bonded with a metallic or metallized structure. With some examples at least one of the first and second electrical conductor paths may be provided on a flexible substrate, such as a flexible foil adhesively attached or bonded on the first elongated device component. With other examples at least one of the first and the second electrical conductors is insert molded in the body, which may be implemented or manufactured as an injection molded plastic component.

With a further example, the supply interface of the electrical power supply device comprises a first counter supply contact electrically connected to the rechargeable energy reservoir and electrically connectable to a complementary shaped first supply contact of the drug delivery device. The electrical supply device and hence the supply interface further comprises a second counter supply contact electrically connected to the rechargeable energy reservoir and electrically connectable to a complementary shaped second supply contact of the drug delivery device. The first and the second supply contacts may get in direct surface contact with the complementary shaped first and second supply contacts of the drug delivery device when the electrical power supply device is mechanically connected to the drug delivery device, in particular to a housing of the drug delivery device.

The direct mutual surface contact between the first counter supply contact and the first supply contact as well as the direct surface contact between the second counter supply contact and the second supply contact provides a transfer of electrical energy across the mutually engaged interfaces of the electrical power supply device and the drug delivery device, i.e. when the supply interface is appropriately engaged with the electrical interface of the drug delivery device.

According to a further example, the first counter supply contact is connected to or is integrated into a proximal end of the first electric supply conductor path. Also, the second counter supply contact is connected to or is integrated into a proximal end of the second electric supply conductor path. In other words, a proximal end of the first and/or of the second electric supply conductor path(s) is provided or terminates with the first and second counter supply contacts, respectively. In order to provide a good and reliable electrical connection across the supply interface engaged with the electrical interface it is of particular benefit, when at least one of the first counter supply contact and the supply contact comprises a circumferentially widened portion relative to the circumferential extent of the first or second electric supply conductor paths. In this way there can be provided a durable and reliable electrical connection between the first and second counter supply contact with complementary shaped first and second supply contacts even if a position or orientation of the body of the electrical power supply device should be subject to some geometric tolerances relative to the position or orientation of the housing or of a housing portion of the drug delivery device.

According to a further example, the first counter supply contact and the second counter supply contact are arranged at the proximal end of the body. This way, there is provided a rather good accessibility for the first and second counter supply contacts, thus enabling a rather easy and straightforward electrical connection with the supply contact of the electrical interface of the drug delivery device.

According to a further example, the first counter supply contact and the second counter supply contact are arranged or integrated onto the mechanical counter interface. In this way, the mechanical counter interface provides a twofold function. First, the mechanical counter interface provides a mechanical fastening of the body of the electrical power supply device to the drug delivery device, e.g. to a housing thereof. Second, by integrating the first and/or the second counter supply contact into the mechanical counter interface there may be simultaneously provided an electrical connection between the supply interface and the electrical interface as the mechanical counter interface engages with the mechanical interface of the drug delivery device.

With some examples, engaging of the mechanical interface with the mechanical counter interface is accompanied by a feedback signal, e.g. by an acoustically or haptically perceivable feedback. Receiving such a feedback is a direct indication for a user that not only the mechanical connection is established between the electrical power supply device and the housing of the drug delivery device but that also the supply interface of the electrical power supply device is electrically connected with the electrical interface of the drug delivery device.

According to a further example, the first and the second counter supply contacts are arranged on or in the proximal end of the sidewall of the body of the electrical power supply device. They may be even provided at a proximal end face of the sidewall of the body and may longitudinally and/or radially engage or connect with the complementary shaped first and second supply contacts as provided on or in the drug delivery device when the electrical power supply device is appropriately assembled or mechanically connected to the drug delivery device.

According to a further example, the first and the second counter supply contacts are arranged at a circumferential offset from each other on or in the sidewall of the body of the electrical power supply. When the body of the electrical power supply is implemented as an injection molded plastic component, the material of the body may provide an inherent electrical isolation between the first and the second counter supply contact.

With a further example, the first counter supply contact and the second counter supply contact are arranged at a longitudinal offset from each other on or in the sidewall. Such a longitudinal offset may be advantageous so as to avoid a cross coupling, namely of the first counter supply contact getting in contact with the second supply contact. Accordingly, the supply contacts as provided on or in a housing of the drug delivery device may be also provided at a longitudinal offset from each other. This way and independent of a rotational position or rotational orientation of the body of the electrical power supply with regards to the longitudinal axis as an axis of rotation, there can be always provided a well-defined mutual electrical contact between the first supply contact only and exclusively with the first counter supply contact. Likewise, there can be provided an exclusive electrical contact between the second supply contact at the second counter supply contact.

With still another example, the first counter supply contact and the second counter supply contact are integrated into the counter fastener. There may be provided even a first and a second counter fastener to engage with complementary shaped first and second fasteners of the mechanical interface of the drug delivery device. Here, the first counter supply contact may be integrated into the first counter fastener and the second counter supply contact may be integrated into the second counter fastener. On the side of the drug delivery device a respective implementation is provided.

By implementing the first and the second counter supply contacts in at least one counter fastener and by integrating respective first and second supply contacts on the side of the drug delivery device in the complementary shaped fastener there will be automatically provided an electrical contact between the first supply contact and the first counter supply contact as well as between the second supply contact and the second counter supply contact as the fastener engages the counter fastener.

The integration of electrical contact elements into a mechanical fastening structure of the electrical power supply device is of particular benefit to establish and to maintain an electrical connection between the rechargeable energy reservoir and the electronic unit as soon and as long as the electrical power supply device is appropriately mounted or mechanically connected to the drug delivery device.

According to a further example, the electrical supply device comprises a logic circuit. The logic circuit comprises a processor connected to the rechargeable energy reservoir and being further connected to at least one of the supply interface and the charging interface. The processor is operable to control at least one of charging the rechargeable energy reservoir via the charging interface and to provide electrical energy to the drug delivery device via the supply interface.

The logic circuit, hence its processor may be operable to control depletion of the rechargeable energy reservoir, e.g. by providing electrical energy to the drug delivery device. It may be further operable and configured to control recharging of the rechargeable energy reservoir when the charging interface is electrically connected to the external power supply. In this way, there can be provided a rather intelligent power management of the rechargeable energy reservoir.

According to another example, the electrical supply device further comprises a timer and a signal generator controllable by the processor. The timer and the signal generator as well as the processor may be implemented or integrated into or on a common printed circuit board. Here, the processor is operable to start the timer in response to detect an electrical disconnection or electrical coupling of the supply interface from the electronic unit of the drug delivery device. The processor is further operable to stop the timer in response to detect an electrical reconnection of the supply interface to the electronic unit of the drug delivery device.

Furthermore, the processor is operable to cause the signal generator to generate an out of time alert signal when the count value of the timer exceeds a predefined maximum count value. In this way, and when the electrical supply device is for instance implemented as a protective cap of a handheld drug delivery device, the timer may be automatically started by the processor when the processor detects a disconnection of the supply interface from the electronic unit, hence from the electrical interface of the drug delivery device. Such a disconnection typically takes place when the protective cap is detached from the drug delivery device.

Hence, upon detaching of the protective cap from the drug delivery device the processor of the electrical supply device starts the timer. When the protective cap is in reattached or reassembled to the drug delivery device the resulting reconnection of the supply interface to the electrical interface of the drug delivery device and hence to the electronic unit of the drug delivery device can be detected by the processor in the same or in a like manner. Then, the timer may be stopped by the processor and may be optionally reset.

The processor is further coupled with the signal generator and is operable to generate an out of time alert signal when the count value of the timer exceeds a predefined maximum count value. Exceeding a predefined maximum count value may occur, when the protective cap remains detached from the drug delivery device for an inadmissible large time interval. Then, and by causing the signal generator to generate an out of time alert signal the user is warned or provided with a respective information that reconnection of the protective cap to the drug delivery device is due or even overdue.

The signal generator may be implemented in many different ways. It may be implemented as one of a visual signal generator, as an acoustic signal generator or as a signal generator operable to generate a haptically perceivable signal. In any way, the signal generator is operable to generate a user-perceivable alert signal so as to attract the user's attention and to prompt the user to put the protective cap and hence the electrical supply device back onto the drug delivery device.

According to another example, the processor is operable to determine a charging level of the rechargeable energy reservoir. Here, the processor is hence operable to cause the signal generator to generate an energy depletion alert signal when the charging level drops below a predefined minimum charging threshold. The minimum charging threshold may be selected such that the remaining electrical energy provided in the rechargeable energy reservoir is sufficient to charge the local battery of the electronic unit at least one more time. Yet with some examples the minimum charging threshold may be set to enable a repeated recharging, hence, an at least twofold charging of the local battery of the electronic unit of the drug delivery device.

In any way and when the processor detects that the rechargeable energy reservoir runs out of electric energy a respective energy depletion alert signal is generated, thus prompting the user to connect the electrical power supply device with the external power supply so as to charge the rechargeable energy reservoir. In this way there can be avoided a complete depletion of the rechargeable energy reservoir.

Typically, the energy depletion alert signal is at least one of a visual signal, an acoustic signal and a haptic signal. The energy depletion alert signal may distinguish from the out of time alert signal. For instance, the energy depletion alert signal may be provided as an acoustic signal, such as a frequent beep noise or the like. Alternatively, the energy depletion alert signal may be provided in form of a blinking or durable light.

According to another aspect, the present disclosure relates to a drug delivery device configured for setting and dispensing of a dose of a medicament. The drug delivery device comprises a housing, an electronic unit arranged on or inside the housing. The drug delivery device further comprises an electrical interface on or inside the housing. The electrical interface is electrically connected to the electronic unit and is configured to electrically couple to a supply interface of an electric supply device as described above.

When the drug delivery device is implemented as a handheld injection device, such as a pen-type injector, the electrical interface may be provided on an outside surface of a housing of the drug delivery device. It may be provided at a distal end of a proximal housing part. Alternatively, it may be provided at a proximal end of a distal housing part, e.g. implemented as a cartridge holder. In particular, the electrical interface of the drug delivery device may engage with the supply interface of the electrical power supply device, which, when implemented as a protective cap of the drug delivery device, is provided at or near a proximal end of the protective cap.

In this way, the electrical interface may be provided on the outside surface of a proximal end of a cartridge holder or on a distal end face or on an outside surface of a distal end of a body of the housing of the drug delivery device longitudinally adjoining the cartridge holder.

According to a further example, the drug delivery device comprises a mechanical interface and further comprises the electrical supply device as described above. The electrical supply device comprises a mechanical counter interface to detachably fasten to the mechanical interface of the drug delivery device.

Typically, and when the mechanical counter interface of the electrical supply device engages the mechanical interface of the drug delivery device the supply interface of the electrical power supply device is in electrical contact and is hence electrically coupled to the electrical interface of the drug delivery device. Since the electrical interface of the drug delivery device is electrically connected to the electronic unit, the rechargeable energy reservoir of the electrical power supply device may be then also appropriately electrically connected to the electronic unit.

The rechargeable energy reservoir of the electrical power supply device may be then used to recharge or to charge a local battery of the electronic unit of the drug delivery device. Detaching of the electrical power supply device disconnects the rechargeable energy reservoir of the electrical power supply device from the electronic unit of the drug delivery device. Then, the electronic unit of the drug delivery device may be operated standalone on the basis of electrical energy as provided by the local battery of the electronic unit.

According to another example, the drug delivery device comprises a drive unit, e.g. comprising a piston rod movable along the longitudinal direction. The drug delivery device further comprises a medicament container filled with a medicament. The drive unit, e.g. the piston rod, is configured to operably engage with the medicament container in order to expel or to withdraw a dose of the medicament from the medicament container.

The drug delivery device as described above is configured to interact with the electrical power supply device as described above. Insofar, all features, benefits as described above in connection with the electrical power supply device equally apply to the drug delivery device; and vice versa.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes.

Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, numerous examples of a method of authenticating a user to use a drug delivery device as well as numerous hardware configurations including an authentication system, an electronic circuit, a drug delivery device, a supplementary device and numerous implementations of mobile electronic devices will be described in greater detail by making reference to the drawings, in which:

FIG. 1 schematically illustrates a longitudinal cross-section through an example of a drug delivery device,

FIG. 2 shows an enlarged view of the drive unit of the drug delivery device of FIG. 1,

FIG. 3 is an enlarged view of a portion of first and second device components,

FIG. 4 is an enlarged view of an electromechanical actuator in an unlocked or released configuration,

FIG. 5 shows the electromechanical actuator when actuated,

FIG. 6 is a diagram of a voltage in at least one of the electrical conductor path and/or in the electrical control path during a typical scenario of use,

FIG. 7 shows a further example of the drug delivery device with a protective cap provided with an energy reservoir,

FIG. 8 is an enlarged view of the distal end of the protective cap according to FIG. 7,

FIG. 9 shows an example of an interface and a counter into interface for fastening the protective cap to the drive unit or to the drug delivery device,

FIG. 10 shows an example of an interface with electrical contact elements and counter contact elements,

FIG. 11 shows another example of the interface according to FIG. 10,

FIG. 12 shows a further example of the interface according to FIG. 10,

FIG. 13 shows a further example of an interface of FIG. 10,

FIG. 14 shows a further schematic example of a conducting or supply path provided across the interface and the counter interface,

FIG. 15 shows a transverse cross-section through an example of an interface connected with a counter interface,

FIG. 16 shows an example of a counter interface provided at a proximal end of a container unit configured for fastening to the first interface of the drive unit,

FIG. 17 shows the interfaces according to FIG. 16 when engaged,

FIG. 18 shows another example of an interface and a counter interface according to FIG. 16,

FIG. 19 shows the interfaces of FIG. 18 when mutually engaged,

FIG. 20 shows a cross-section A-A before or during engagement of the interfaces and

FIG. 21 shows a cross-section A-A when the interfaces are mutually engaged,

FIG. 22 is illustrative of numerous examples of counter interfaces of different container units configured for fastening with the first interface of the drive unit,

FIG. 23 shows a cross-section through the first interface of the drive unit,

FIG. 24 is a block diagram of the drug delivery device configured to communicate with an external electronic device,

FIG. 25 shows another example of a first interface of the drive unit engaged with a counter interface of the container unit,

FIG. 26 is an enlarged illustration of the interfaces before or during mutual assembly or engagement,

FIG. 27 shows the example of FIG. 26 when the interfaces are engaged or connected,

FIG. 28 shows another example of the first interface for operating with a counter interface of a container unit before or during mutual assembly and

FIG. 29 shows the interfaces according to FIG. 28 when in a final assembly configuration,

FIG. 30 shows another example of a drive unit in longitudinal cross-section,

FIG. 31 shows a cross-section B-B according to FIG. 30,

FIG. 32 shows an enlarged view of the cross-section of FIG. 31,

FIG. 33 is a block diagram of the electrical power supply device,

FIG. 34 is a flowchart of a method of operation of the electrical power supply device and

FIG. 35 is a flowchart of a further method of operating the electrical power supply device.

### DETAILED DESCRIPTION

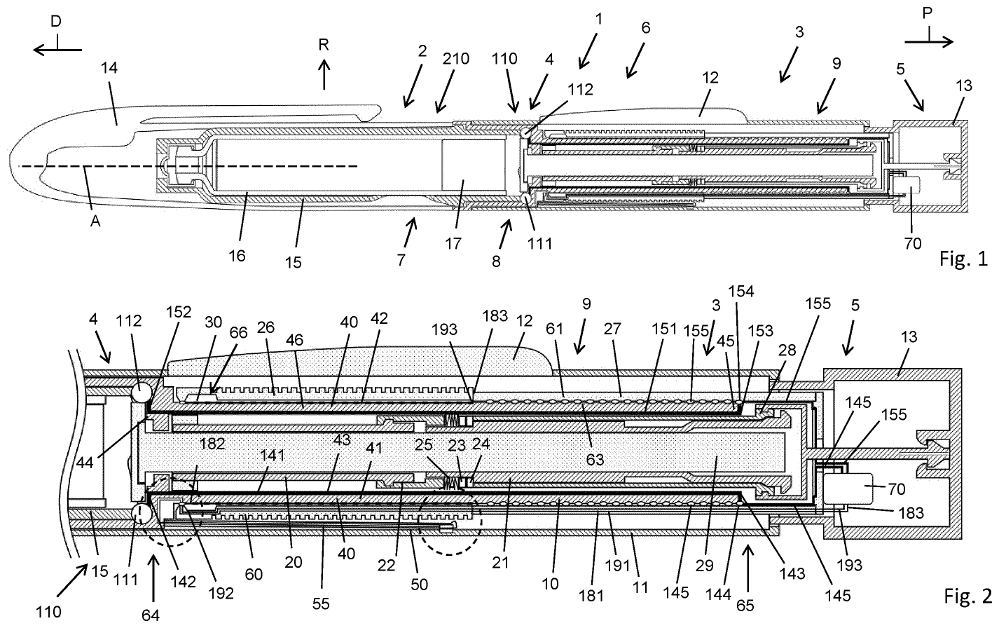

FIGS. 1, 2, 7 and 30 show exemplary embodiments of a drug delivery device 1 in a cross-sectional view. The drug delivery device 1 is a variable dose device, in which different doses of a drug to be dispensed can be set or dialed, respectively, by a user. The drug delivery device is a dial extension pen.

FIG. 1 also indicates the coordinate system used herein for specifying positions of members or elements or features. The distal direction D and proximal direction P run parallel to the longitudinal axis A. The longitudinal axis A is a main extension axis of the device 1. The radial direction R is a direction perpendicular to the longitudinal axis A and intersecting with the longitudinal axis A. A tangential direction, also referred to as circumferential direction is a direction perpendicular to the radial direction R and to the longitudinal axis A.

The drug delivery device 1 comprises a drive unit 3 with a setting mechanism and a dispense mechanism. The setting mechanism is configured for setting a drug dose and the dispense mechanism is configured for dispensing a drug dose. The functional principles of the mechanisms are explained further below.

The drive unit 3 comprises an inner body 10 and a housing element 11, in the following also referred to as outer body 11. The inner body 10 and the outer body 11 are fixedly connected to each other, i.e. they cannot be rotated or moved axially with respect to each other. The outer body 11 forms an outer surface of the drug delivery device 1 which can be touched or grabbed by a user.

The drug delivery device 1 further comprises a protective cap 14 and a user interface member 13 in form of a knob 13. The knob 13 is a dose setting member configured to be operated by a user for setting a drug dose. At the same time, the knob 13 is dose dispense member configured to be operated by a user in order to dispense a drug dose.

A drug reservoir unit 2, also referred to as a container unit 2, comprises a reservoir 16 and a reservoir holder 15 is received within the cap 14. The reservoir 16 is filled with a drug or medicament. The reservoir 16 is sealed in proximal direction P by a stopper 17.

The reservoir unit 2 is operatively coupled or connected, respectively, to the drive unit 3. The drive unit 3 is configured to enable a dispense process for dispensing a drug dose by acting on the drug reservoir 16. For dispensing a drug dose, the stopper 17 is pushed in distal direction D by a plunger rod 29 of the drive unit 3. When an injection needle 18 is connected to drug reservoir a dose of the medicament can be injected into biological tissue.

The coupling between the drive unit 3 and the reservoir unit 2 is realized by the inner body 10 being coupled to the reservoir holder 15 via a connection interface which might be a snap connection or a threaded connection. The coupling is preferably reversible. For example, the container unit 2 is axially and rotationally fixed to the inner body 10 by the coupling.

The drive unit 3 further comprises a number sleeve 26 and a dial sleeve 27 which are fixedly coupled to each other (e.g. they cannot rotate or move axially relative to each other). The dial sleeve 27 may comprise an inner thread which is engaged with an outer thread of the inner body 10. On an outer surface of the number sleeve 26, numbers may be shown. The user can see the numbers through a window 12. The window 12 may comprise a lens. The window 12 is formed in the outer body 11. The numbers visible in the window 12 indicate to a user the set/dialed dose. Due to the threaded coupling between the dial sleeve 27 and the inner body 10, the dial sleeve 27 and the number sleeve 26 are moved on a helical path in proximal direction relative to the body 10, 11 during setting a drug dose and dispensing a drug dose as will be explained further below.

The drive unit 3 also comprises a drive sleeve. The drive sleeve comprises a distal drive sleeve 20, a proximal drive sleeve 21 and a drive sleeve coupler 22 coupling the distal drive sleeve 20 to the proximal drive sleeve 21. For setting a drug dose and dispensing a drug dose, the distal drive sleeve 20 and the proximal drive sleeve 21 are fixedly coupled to each other via the drive sleeve coupler 22 so that these elements can neither rotated nor move axially relative to each other. The distal drive sleeve 20 may comprise an inner thread which is engaged with an outer thread of the plunger rod 29. An outer thread of the distal drive sleeve 20 may be engaged to an inner thread of a last dose nut 30, the function of which will be explained further below.

Furthermore, the drive unit 3 comprises a clutch 28, which is fixedly coupled to the knob 13 so that, during setting a drug dose and dispensing a drug dose, the clutch 28 and the knob 13 are not rotated or moved axially relative to each other. The clutch 28 is coupled to the proximal drive sleeve 20 via a splined engagement. This splined engagement may allow a certain axial movement of the clutch 28 relative to the proximal drive sleeve 21 but does not allow a relative rotation between these two elements.

A distal clicker 23, a proximal clicker 24 and a clutch spring 25 of the drive unit 3 are arranged between the clutch 28 and the drive sleeve coupler 22. The clutch spring 25 is coupled to the drive sleeve coupler 22 and to the distal clicker 23. The distal clicker 23 is configured to abut against the proximal clicker 24 in proximal direction P. The proximal clicker 24 is configured to abut against the clutch 28 in proximal direction P. Thus, the clutch spring 25 is configured to bias the distal clicker 23, the proximal clicker 24 and the clutch 28 in proximal direction P relative to the drive sleeve coupler 22.

The distal clicker 23 may be permanently splined to the proximal drive sleeve 21 so that a relative rotation between these two elements is prevented. However, a certain axial movement between the distal clicker 23 and the proximal drive sleeve 21 may be allowed. The proximal clicker 24 may be permanently splined to the inner body 10 so that a relative rotation between these two elements is prevented, whereas a certain relative axial movement may be allowed.

The distal face of the clutch 28 and the proximal face of the proximal clicker 24 may both be toothed so that these two faces may engage into each other. Furthermore, the distal face of the proximal clicker 24 and the proximal face of the distal clicker 23 may both be toothed so that these two toothed faces can engage into each other. A proximal face of the clutch 28 may be toothed, e.g. dog toothed, and may be arranged to engage a toothed, e.g. dog toothed, distal face of the dial sleeve 27.

FIG. 1 shows the drug delivery device 1 when no dose is set (0 units/0 unit position). Dose setting may be allowed in discrete units of 1, e.g. from 0 to 80 units. For setting a desired drug dose, the user has to rotate the knob 13. This is done without pressing on the knob 13 in distal direction D. As long as one does not press on the knob 13 in distal direction D, a dog toothed engagement between the clutch 28 and the dial sleeve 27 is established due to the clutch spring 25 either biasing the clutch 28 in proximal direction P or at least preventing the clutch 28 from moving in distal direction D on its own. The dog toothed engagement between the clutch 28 and the dial sleeve 27 has as a consequence that the two elements are rotationally locked to each other so that, when the knob 13 is rotated, also the dial sleeve 27 and the number sleeve 26 are rotated. Since the dial sleeve 27 is threadedly engaged with the inner body 10, rotating the knob 13 has as a consequence that the knob 13, the clutch 28, the dial sleeve 27 and the number sleeve 26 move on a helical path in proximal direction P relative to the body 10, 11. Thereby, the numbers of the number sleeve 26 visible through the window 12 increase.

Since the proximal drive sleeve 21 is splined to the clutch 28, also the proximal drive sleeve 21 and with it the distal drive sleeve 20 and the drive sleeve coupler 22 are moved on the helical path in proximal direction P relative to the inner body 10.

The plunger rod 29 comprises two outer threads with opposite hand which overlap with each other. The plunger rod 29 is threadedly engaged with the inner thread of the distal drive sleeve 20. The threads are chosen such that during the helical movement of the distal drive sleeve 20 in proximal direction P, the plunger rod 29 does not rotate and is also not moved axially.

The last dose nut 30 may be splined to the inner body 10 and, therefore, cannot rotate relative to the inner body 10. Due to the threaded engagement of the last dose nut 30 with the distal drive sleeve 20, the last dose nut 30 is forced to move in proximal direction P during setting a drug dose. When the maximum dose has been set (e.g. 80 units—independently of whether it has been set in only one drug setting process or several drug setting processes), the last dose nut 30 establishes a rotation-lock interface with the distal drive sleeve 20 so that the last dose nut 30 can no longer rotate relative to the distal drive sleeve 20. As a consequence of this, the distal drive sleeve 20 can no longer be rotated and no further drug dose can be set. The drug delivery device 1 then has to be reset to its initial state.

During setting a drug dose, the toothed faces of the distal clicker 23 and the proximal clicker 24 facing each other ratchet over each other thereby creating a click sound which indicates to a user that a drug dose is set. For this purpose, the teeth of the two faces are preferably formed as shallow triangles so that relative rotation between the clickers 23 and 24 is possible leading to a repeated slight compression and decompression of the clutch spring 25.

After the desired dose has been set, the user can now press on the knob 13 in distal direction D in order to dispense the set drug dose. Thereby, the distally directed force on the knob 13 is transferred from the knob 13 via the clutch 28 to the proximal clicker 24, from there to the distal clicker 23 and this compresses the clutch spring 25. The two clickers 23 and 24 are now pressed against each other and their toothed faces are engaged. Relative rotation between the two clickers 23, 24 is now prevented. Since the proximal clicker 24 is splined to the inner body 10 and the distal clicker 23 is splined to the proximal drive sleeve 21, the proximal drive sleeve 21 can no longer rotate relative to the inner body 10. However, since the proximal drive sleeve 21 is also splined to the clutch 28, also the clutch 28 and the knob 13 can no longer rotate relative to the inner body 10.

The distally directed force applied to the knob 13 has as a consequence that the clutch 28 together with the knob 13 slightly moves in distal direction D relative to the dial sleeve 27 so that the clutch spring 25 is compressed, as already mentioned. The dog toothed engagement between the dial sleeve 27 and the clutch 28 is thereby released so that the dial sleeve 27 is no longer rotationally locked to the clutch 28. Therefore, when the knob 13 is pressed in distal direction D, the dial sleeve 27 together with the number sleeve 26 can still rotate relative to the inner body 10. When the knob 13 is now moved in distal direction D, a stop against the dial sleeve 27 forces the dial sleeve 27 to also move in distal direction D. Due to the threaded engagement of the dial sleeve 27 with the inner body 10, the dial sleeve 27 together with the number sleeve 26 moves on a helical path in distal direction D. Thereby, the numbers of the number sleeve 26 visible in the window 12 decrease.

At the same time, the clutch 28, the clickers 23, 24 and the drive sleeve 20, 21, 22 are forced to move in distal direction D (without rotation). The threaded engagement between the plunger rod 29 and the distal drive sleeve 20 forces the plunger rod 29 to rotate. A further threaded engagement between the plunger rod 29 and an inner thread of the inner body 10 may then force the plunger rod 29 to also move distally in order to push the stopper 17 inside the cartridge 16 in distal direction D for dispensing the set drug dose. Since the distal drive sleeve 20 is not rotate during dispensing, the last dose not 30 moves together with the distal drive sleeve 20 in distal direction D without changing its position relative to the distal drive sleeve 20.

After having dispensed the set drug dose and when the knob 13 has been completely moved back into its initial position, a new drug dose may be set by again rotating the knob 13 on a helical path in proximal direction P. During this, the plunger rod 29 does not change its position. Only when dispensing a dose, the plunger rod 29 is moved in distal direction D.

As explained with respect to FIG. 1, one user interface member in form of a knob 13 is used for setting a drug dose as well as for dispensing the drug dose. However, it is also possible to use separate user interface members for setting and dispensing a drug dose.

Further examples of the drive unit 3 to be implemented with the injection device 1 can be found in WO 2014/033195 A1 or WO 2014/033197 A1 the entirety of which being incorporated herein by reference.

FIGS. 2-33 show the drug delivery device 1 of FIG. 1 but in different views than FIG. 1 and with more details. FIG. 2 only shows the proximal part of the drug delivery device 1 in order to better illustrate some of the details.

The drug delivery device 1 is provided as an all-mechanically implemented pen-type injection device. Accordingly, a force required for injecting the medicament is entirely provided by a user of the device. The drug delivery device 1 may be implemented as a disposable device. Such devices are intended to be discarded when the medicament provided in the drug container 16 has been used up. With other examples the drug delivery device is implemented as a reusable device. Here, the drive unit 3 forming or constituting the proximal portion of the drug delivery device is provided with a connection interface 8 that provides a mechanical releasable connection with the container unit 2. The drug delivery device may further comprise a further connection interface 7 by way of which the protective cap 14 is detachably fixable to at least one of the housing 6, e.g. the drive unit housing 9 and the cartridge holder 15. Generally, the housing 6 of the drug delivery device comprises a proximally located drive unit housing 9 connected or connectable with the container unit 2. The container unit 2 comprises the cartridge holder 15 serving as a distal housing component of the drug delivery device 1. The cartridge holder 15 is typically to be covered by the protective cap 14 when the drug delivery device is not in use.

As shown in greater detail in FIG. 2 the drive unit 3 is provided with an electronic unit 70. The electronic unit 70 is arranged inside a hollow space covered by the dose button 13. It is provided near or at the proximal end 5 of the drive unit 3. At or near an oppositely located distal end the drive unit 3 comprises a first interface 110. The first interface 110 is provided with an electrical contact element 111 and a second electrical contact element 112. The first and the second electrical contact elements 111, 112 may be provided inside a receptacle at the distal end 4 of the drive unit 3. The receptacle may be sized and configured to receive a complementary shaped insert section of the cartridge holder 15.

The first electrical contact element 111 is connected with a first electrical conductor path 141, which is located on or inside a first elongated device component 40. With the presently illustrated example the first elongated device component 40 is provided by the inner body 10 of the drive unit 3. Alternatively, it may be provided by any other elongated component of the drive unit 3 as described above.

The first device component 40 comprises a somewhat tubular and elongated shape. As illustrated, the first electrical conductor path 141 extends along an inside surface 43 of the sidewall 41 of the first device component 40. Likewise, there is provided a second electrical conductor path 151. Also the second electrical conductor path 151 is provided on the inside surface 43 of the sidewall 41 of the first elongated device component 40.

The first and the second electrical contact elements 111, 112 are provided at a distally facing longitudinal end face 44 of the first housing component. Here, the longitudinal ends, i.e. the distal ends 142, 152 of the first and second electrical conductor paths 141, 151 are in permanent electrical contact with the first and second electrical contact elements 111, 112, respectively. At the opposite longitudinal end the first elongated device component 40 comprises a proximally facing longitudinal end face 45.

A proximal end 143, 153 of the first and of the second electrical conductor paths 141, 151 terminates at the respective longitudinal end face 45. As illustrated in FIG. 2, the proximal ends 143, 153 extend radially outwardly at the longitudinal end face 45 of the first device component 40.

The drive unit 3 further comprises at least a second device component 60, presently implemented as the dial sleeve 27 and/or as the number sleeve 26. The second device component 60 is also of tubular shape. Alternatively and generally, the second device component may be represented by any component of the drive unit 3 as described above, which is movable relative to the first device component 40.

The first device component 40 is located inside the second device component 60. The tubular-shaped sidewall 61 of the second device component 60 encloses an outside surface 42 of the first device component 14. Moreover, the first device component 40 and the second device component 60 are threadedly engaged. Accordingly, the outside surface 42 of the first device component 40 is provided with a threaded section 46 in threaded engagement with a correspondingly shaped threaded section 66 at the inside surface 63 of the second device component 60. In this way, the second device component 60 is movable relative to the first device component 40 along a helical path.

On the inside of the sidewall 61 of the second device component 60, e.g. towards a proximal end of the second device component 60, e.g. formed by the dial sleeve 27, there is provided a first conductor path extension 145 and a second conductor path extension 155. The first and the second conductor path extensions 145, 155 are provided on the side surface 63 of the sidewall 61 of the second device component 60.

The first conductor path extension 145 and the second conductor path extension 155 comprise a helically wound structure with numerous windings. The helically wound structure of the first conductor path extension 145 and the helically wound structure of the second conductor path extension 155 may be arranged in a nested or convoluted manner on the inside surface 63 of the sidewall 61 of the second device component 60. Towards the proximal end 65 the first and second conductor path extensions 145, 155 are rather straight shaped and are further electrically connected to the electronic unit 70.

As illustrated in FIG. 2, the proximal end 143 of the first electrical conductor path 141 is in sliding contact with the first conductor path extension 145. Details of the sliding contact 144 are further illustrated with regards to FIGS. 31 and 32. Likewise, the proximal end 153 of the second electrical conductor path 153 is provided with a sliding contact 154 being in surface contact with the second conductor path extension 155.

The helical structure of the first and the second conductor path extensions 145, 155 has the same lead as the threaded engagement between the first device component 40 and the second device component 60. In this way and when for instance the second device component 60 is subject to a helical motion relative to the first device component 40 the electrical contact between the first electrical conductor path 141 and the first electrical conductor path extension 145 is maintained. The same applies for the second conductor path 151 and the second conductor path extension 155.

In FIGS. 2-5 there is further illustrated a first electrical control path 181 and a second electrical control path 191. The first and second electrical control paths 181, 191 extend from the electronic unit 70 towards an electromechanical actuator 50 provided at a longitudinal distance from the proximal end 5 of the drug delivery device 1. The first electrical control path 181 comprises a proximal end 183 permanently connected to the electronic unit 70. Likewise, the second electrical control path 191 comprises a proximal end 193 permanently connected to the electronic control unit 70. The electronic control unit 70 is arranged or mounted at a proximal end of the second device component 60. It may be located at a proximal end face of the second device component 60.

The first electrical control path 181 and the second electrical control path 191 are located on or inside the second device component 60. They extend in longitudinal direction along or in the sidewall 61 of the second device component 60. As illustrated in FIGS. 2-5 the electromechanical actuator 50 is provided on or in the inner body 10. It may be integrally formed with the first device component 40. The electromechanical actuator 50 may comprise a bending arm 55 extending in the longitudinal direction A. It comprises a free end towards or at its proximal end. The proximal end or free end of the bending arm for 55 is provided with a radially inwardly extending protrusion 53 to engage with a complementary-shaped recess 54 provided on the second device component 60.

The electromechanical actuator 50 comprises a first magnet 51, e.g. provided on or inside the outer body 11 as illustrated in FIGS. 4 and 5. The electromechanical actuator 50 further comprises a second magnet 52 in longitudinal alignment with the magnet 51. The second magnet 52 may be implemented as an electromagnet. It is electrically connected with a first control path extension 185 and a second control path extension 195. By providing a current via the first and the second control path extensions 185, 195 the second magnet 52 generates a magnetic field, e.g. interacting with the static magnetic field of the first magnet 51 thus leading to an attraction or repelling between the first and the second magnets 51, 52.

The bending arm 55 is resiliently deformable in radial direction. By suitably driving the electromagnet 52 with a driving current there can be induced a radially directed movement of the bending arm 55 and hence of the protrusion 53. In this way and by applying a current to the electromagnet 52 there can be established or abrogated a mechanical engagement between the protrusion 53 and one of the numerous recesses 54 as provided on an outside surface of the second device component 60.

In FIG. 4, an unlocking configuration is illustrated, in which the bending arm 55 and hence its protrusion 53 is out of engagement from the recess(es) 54. In FIG. 5 actuation or activation of the electromagnet 52 leads to a radially inwardly directed movement of the protrusion 53, thereby engaging the recess 54. Since the bending arm 55 is fixedly attached to the inner body 10 the second device component 60, in particular the number sleeve 26 is locked against rotation. Accordingly, and in the locked configuration as illustrated in FIG. 5 at least one of setting of a dose and injecting of a dose is locked.

With other examples the locking mechanism 56 is activated and hence the protrusion 53 is in engagement with the recess(es) 54 when the electromechanical actuator 50 is deactivated or is in an initial state. Then and per default, the interlock and hence the locking mechanism 56 is activated. Activation of the electromechanical actuator 50, e.g. applying a current to the electromagnet 52 then leads to an unlocking of the locking mechanism 56.

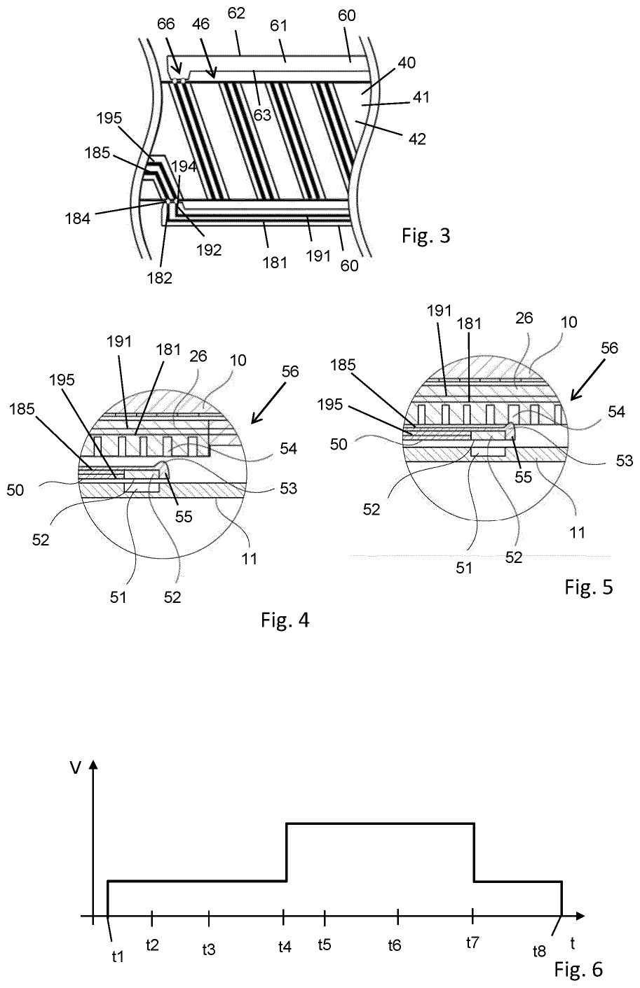

A control signal for activating or deactivating the electromagnet 52 is generated and provided by the electronic unit 70. The control signal is transmitted via the first and the second electrical control paths 181 and the mutual electrical contact configuration between the first electrical control path 181 and the first control path extension 185 as well as via the second electrical control path 191 and the second control path extension 195 as illustrated in greater detail in FIG. 3.

The first and second control path extensions 185, 195 each comprise a proximal end connected to the electromagnet 52 provided on the bending arm 55. The first and second control path extensions 185, 195 may be provided on or inside the bending arm 55. They are electrically isolated from each other. As illustrated in FIG. 2 and towards the distal end of the bending arm 55 the first and second control path extensions 185, 195 extend towards and onto the outside surface 42 of the sidewall 41 of the first elongated device component 40, which may be integrally formed with the bending arm 55.

As illustrated in FIG. 3, the first and second control path extensions 185, 195 are provided in a helical structure on the outside surface 42. The first and second control paths 181, 191 extend on or through the second device component 60, which is in threaded engagement with the first device component 40. As illustrated in FIG. 3, a distal end 182 of the first electrical control path 181 is connected to the first control path extension 185 via a sliding contact 184. Likewise, the distal end 192 of the second electrical control path 191 is connected with the second control path extension 195 of a further sliding contact 194.

The lead of the helical structure of the first and second control path extensions 185, 195 is substantially equal to the lead of the threaded section 66 of the second device component 60 which is in threaded engagement with a complementary shaped threaded section 46 of the first device component 40. In this way and when the second device component 60 is subject to a helical motion relative to the first device component 40, e.g. during setting and/or dispensing of a dose, the electrical contact between the first and second electrical control paths 181, 191 and the respective first and second control path extensions 185, 195 can be maintained.

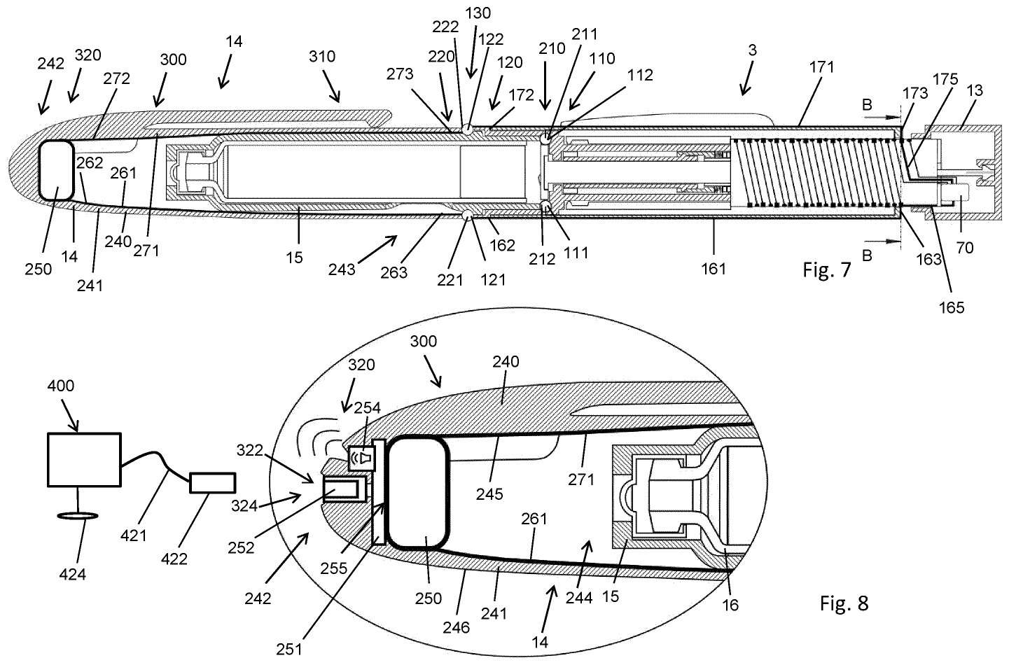

As shown in FIGS. 7 and 8 there is further provided an electrical power supply device 300 comprising a body 240, which in the present example coincides with a body of the protective cap 14. Hence, the electrical power supply device 300 is formed or constituted by the protective cap 14. The body 240 comprises a somewhat tubular-shaped sidewall 241. The sidewall 241 forms or constitutes a receptacle 244 being open towards the proximal direction. The receptacle 244 is sized to receive the cartridge holder 15 of the drug delivery device 1.

The electrical power supply device 300 comprises the supply interface 310 configured to electrically couple or to electrically connect with an electrical interface 130 of the drug delivery device 1. The electrical power supply device 300 further comprises a charging interface 320.

The charging interface 320 and the supply interface 310 are both electrically connected to a rechargeable energy reservoir 250 of the electrical energy supply device 300. The energy reservoir 250 is also implemented as a secondary battery or rechargeable battery.

By way of the charging interface 320. e.g. provided at the distal end 242 of the body 240, the rechargeable energy reservoir 250 can be connected or coupled with an external power supply 400. The distal end 242 of the body 240 comprises a power connector 252 electrically connected to the rechargeable energy reservoir 250. The power connector 252 may comprise a socket 242 configured to engage or to receive a counter plug 422 of the external power supply 400.

As illustrated in FIG. 8 the counter plug 422 may be flexibly and/or movably connected to the external power supply by a cable 421. Alternatively, the electrical connection or coupling between the external power supply 400 and the charging interface 320 may provide a wireless charging. Here, the external power supply 400 may comprise an inductive coupler 424. The power connector 252 may comprise a complementary shaped inductive counter coupler 324 operable to inductively coupled with the inductive coupler 424 when in close vicinity to the external power supply 400.

As further illustrated in FIGS. 7 and 8 the drug delivery device 1 and hence the drive unit 3 is provided with a first electrical supply conductor path 161 and a second electric supply conductor path 171. The drive unit 3 comprises a second interface 120 implemented as a mechanical interface, e.g. provided at the distal end 4 of the drive unit housing 9. The mechanical interface 120 is operable and configured to detachably connect with a mechanical counter interface 220 provided at or near the proximal end 243 of the body 240 of the protective cap 14. Interaction and engagement of the mechanical interface 120 with the mechanical counter interface 220 provides a detachable connection for the protective cap 14 to the housing 6 of the drug delivery device 1.

With the present example, the mechanical interface 120 also comprises an electrical interface 130 configured and operable to electrically engage with the supply interface 310 of the electrical power supply device 300. The supply interface 310 is integrated into the mechanical counter interface 220 of the body 240. In this way and by establishing a mechanical connection between the mechanical interface 120 with the mechanical counter interface 220 there is automatically established an electrical connection between the supply interface 310 and the electrical interface 130.

The electrical interface 130 comprises a first supply contact 121 electrically connected with the first electric supply conductor path 161. The electrical interface 130 further comprises a second supply contact 122 electrically connected to the second electric supply conductor path 171.

The first and the second supply contacts 121, 122 are configured to get into an electrical contact with the complementary shaped supply interface 310 of the electrical power supply device 300. The supply interface 310 comprises first and second counter supply contacts 221, 222 and are provided at a proximal end of a protective cap 14.

In effect, the counter supply contacts 221, 222 are electrically connected with an energy reservoir 250 provided in or on the protective cap 14. The energy reservoir 250, e.g. implemented as a secondary battery is electrically connected to the first counter supply contact 221 via a first electric supply path 261. The second counter supply contact 222 is connected with the energy reservoir 250 by a second electric supply path 271. As illustrated in FIG. 7 a distal end 262 of the first electric supply path 261 is connected with the electric energy reservoir 250. A proximal end 263 is electrically connected to the first counter supply contact 221.

Likewise, a distal end 272 of the second electric supply path 271 is electrically connected to the electric energy reservoir 250.

The first and the second electric supply paths 261, 271 are provided on an inside surface 245 of the sidewall 241 of the hollow receptacle 244 of the body 240. With other examples (not illustrated) the first and the second electric supply paths 261, 271 may be also provided on an outside surface 246 of the sidewall 241 of the body 240.

An oppositely located proximal end 273 of the electric supply path 271 is electrically connected to the second counter supply contact 222. As illustrated in FIG. 7 the first and the second supply path 261, 271 are electrically isolated from each other. They are provided on an inside surface of a sidewall 241 of a cap body 240 of the protective cap 14. The electric energy reservoir 250 is located at or near the distal end 242 of the cap body 240. The counter supply contacts 221, 222 are located at or near the proximal end 243 of the protective cap 14.

When the protective cap 14 and hence when the electrical power supply device 300 is appropriately assembled to the drug delivery device 1 the first electric supply contact 121 is electrically connected to the first counter supply contact 221. Likewise, the second supply contact 122 is electrically connected to the second counter supply contact 222. In this way and since the first and the second electric supply conductor paths 161, 171 are electrically connected to the electronic unit 70, the electronic unit 70 can be provided with electrical energy. In particular, a battery 73 of the electronic unit 70 may be charged by electric energy as provided by the electric energy reservoir 250 of the protective cap 14.

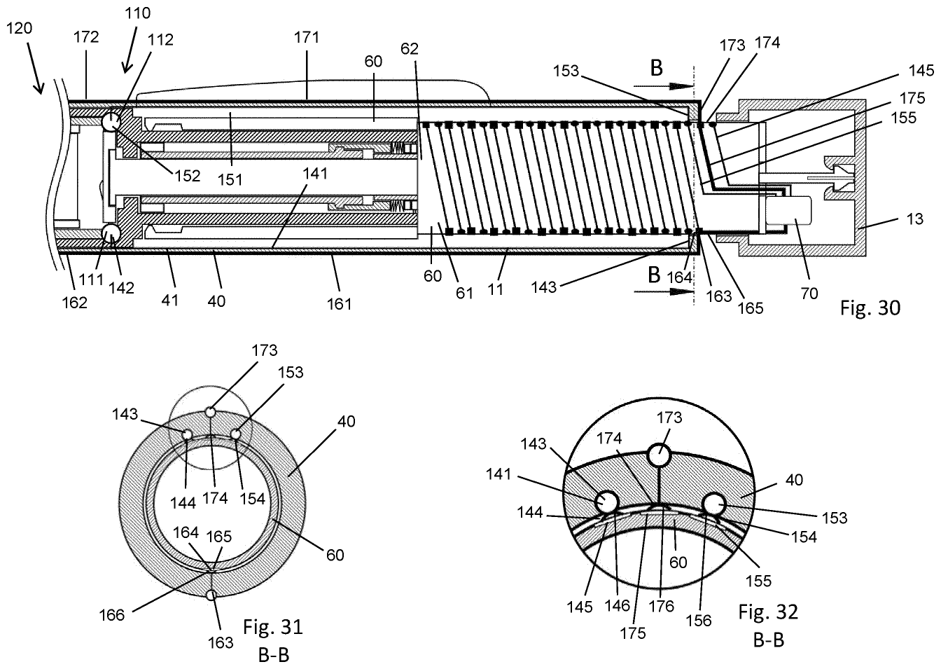

As further illustrated in FIGS. 7 and 30 the first and second electric supply conductor paths 161, 171 are indirectly connected to the electronic unit 70 via the first and second electric supply path extensions 165, 175, the first and second electric supply conductor paths 161, 171 are arranged on an outside surface of the sidewall of the outer body 11. Likewise, they may be provided on an inside surface of the outer body 11. Alternatively, they may be provided on an outside surface of the inner body 10 and hence on an outside surface of the first device component 40.

As illustrated in detail in FIG. 30 the electric supply path extensions 165, 175 are directly electrically connected to the electronic unit 70. They are provided in a helically shaped manner on the outside surface 62 of the second housing component 60. The proximal ends 163, 173 of the first and the second electric supply path 161, 171 terminate at the proximal end of the first device component 40, which is now implemented as the outer body 11.

The proximal end 163 of the first electric supply conductor path 161 is electrically connected with the first electric supply conductor path extension 165 via a sliding contact 164. Likewise, the proximal end 173 of the second electric supply conductor path 171 is electrically connected to the second electric supply conductor path extension 175 by another sliding contact 174. This way there may be provided a permanent electrical contact between the first and second electric supply contact 121, 122 provided at or near a distal end 4 of the drive unit 3 and the electronic unit 70 as provided at or near the proximal end 5 of the drive unit 3 even when the second device component 60 should be subject to a helical motion relative to the first device component 40.

As further illustrated in FIG. 8 the protective cap 14 is provided with a printed circuit board (PCB) 251 and a logic circuit 255. The PCB 251 is further provided with a power connector 252. The power connector 252 provides electrical connection to an external power supply so as to recharge the energy reservoir 250. Typically, the energy reservoir 250 is implemented as a rechargeable battery. The energy reservoir 250 typically comprises an electrical storage capacity that is substantially larger than a respective electrical storage capacity of a battery 73 of the electronic unit 70.

Moreover, there may be provided a signal generator 254 on or in the protective cap and connected to the logic circuit 255. The signal generator 254 is operable to generate and/or to transmit at least one of a visual, audible or a haptic signal. This way, the logic circuit 255 may be configured to autonomously generate an alert signal, e.g. in situations where an abrogated electrical contact with the electronic unit 70 exceeds a predefined time interval. In situations, where the protective cap 14 is not reassembled to the drive unit 3 or to the drug delivery device 1 after use the alert as generated by the signal generator 254 may remind a user of the device to put the protective cap 14 back onto the drug delivery device 1.

Moreover, the electronic circuit 255 may be operable to monitor and/or to detect the charging level of the energy reservoir 250. If the logic circuit 255 should detect that the charging level is below a predefined minimum threshold the logic circuit 255 may be configured to generate a respective alert via the signal generator 254. This way, a user of the device may be prompted to connect the protective cap 14 and hence the entire drug delivery device 1 to an external electric power supply in order to recharge the energy reservoir 250.

With the example of FIG. 30 the first and second electrical conductor paths 141, 151 are located and arranged at an inside of the outer body 11. First and second electric conductor path extensions 145, 155 are provided and arranged on the outside surface 62 of the sidewall 61 of the second housing component 60. Here, the first and second electrical conductor path extensions 145, 155 as well as the first and second electric supply conductor extensions 165, 175 are arranged in a nested or convoluted, hence interleaved manner on the outside surface 62 of the sidewall 61 of the second housing component 60.

Again, a lead of the helical structure of the various conductor path extensions 145, 155, 165, 175 is substantially identical to the lead of the threaded engagement between the first device component 40 and the second device component 60.

In the cross-section according to FIGS. 31 and 32 details of one implementation of a sliding contact 144, 154, 164, 174 are illustrated. Each sliding contact 144, 154, 164, 174 comprises at least one mechanically biased contact conductor 146, 156, 166, 176. With the example of the sliding contact 144 the contact conductor 146 is connected to one of the first electrical conductor path 141 and the first electrical conductor path extension 145. The mechanically biased contact conductor 146 may be implemented as a pliable or elastically deformable conducting tongue that is intrinsically biased in radial direction so as to maintain an electrical contact between the first electrical conductor path 141 as provided on the inside of the first device component 40 and the first electrical conductor path extension 145 as provided on the outside surface of the second device component 60.

With other examples the mechanically biased contact conductor 146 may comprise a conducting pin movable against or under the action of a biasing spring, i.e. a contact spring.

The further contact conductors 156, 166, 176 may be implemented in a likewise manner.

In FIGS. 9-15 there is shown a mechanical interaction between the second interface 120 with the counter interface 220 of the protective cap 14. Here, the mechanical engagement between the interfaces 120, 220 is implemented as snap features or as a snap-type connection. The second interface 120 comprises a fastener 124 implemented as a snap element 125. The snap element 125 comprises a radial protrusion 126 at its distal end longitudinally followed by a recess 127. Accordingly, the counter interface 220 comprises a complementary-shaped counter fastener 224 or a respective counter fastening structure. The counter fastener 224 comprises a complementary-shaped counter snap element 225. The counter snap element 225 comprises a protrusion 227, e.g. a radial protrusion at its proximal end followed by a recess 226. When appropriately assembled the protrusion 227 engages the recess 127 and the protrusion 126 engages the recess 226.

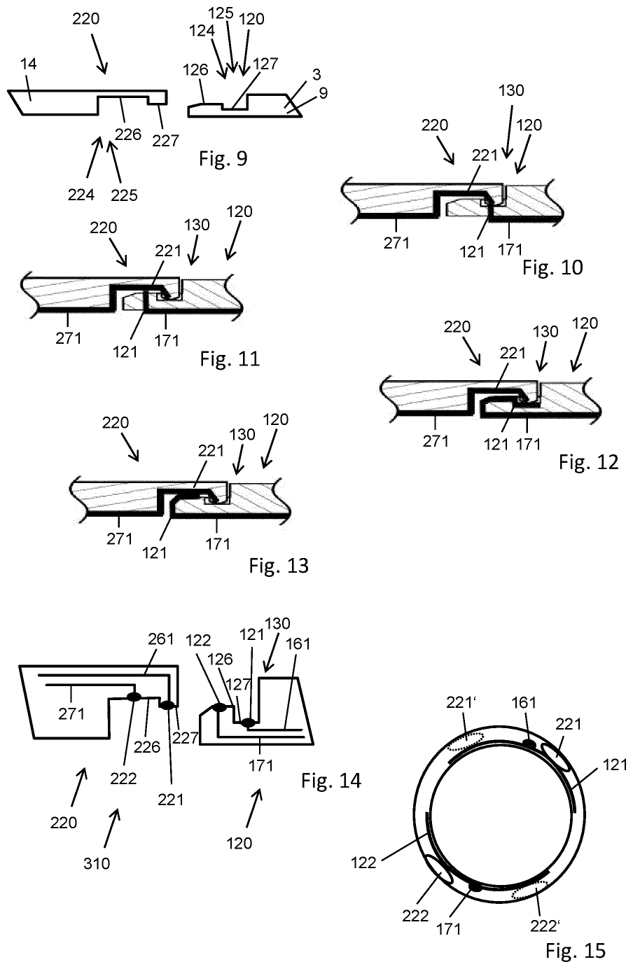

As further illustrated in FIGS. 10-13 the electric supply contact 121 may be provided on an inside of one of the recess 127 and the protrusion 126. Accordingly, the complementary shaped electric counter supply contact 221 is provided on one of the protrusion 227 and the recess 226. With the examples of FIGS. 10 and 12 the electric supply contact 121 terminates in the recess 127. The complementary shaped electric counter supply contact 221 terminates on a respective surface of the protrusion 227.

With the example of FIGS. 11 and 13 the electric supply contact 121 terminates on the protrusion 126. The complementary shaped counter supply contact 221 is provided across the recess 226 and may optionally even extend towards and into the protrusion 227.

In the further illustration of FIG. 14 the respective interfaces 120, 220 are provided with two electric supply contacts 121, 122 and with respective first and second counter supply contact 221, 222. Here, the first electric supply contact 121 is located in the recess 127. The second electric supply contact 122 is provided at a predefined longitudinal distance from the first supply contact 121. It is hence provided on or in the protrusion 126. Accordingly, the first counter supply contact 221 is provided on the protrusion 227 and the second counter supply contact 222 is provided on or in the recess 226. When appropriately connected and upon establishing a snap fit connection between the second interface 120 with the counter interface 220 the first electric supply contact 121 exclusively connected with the first counter supply contact 221. Likewise, the second electric supply contact 122 is in electrical contact with the second counter supply contact 222.

In the cross-section according to FIG. 15 it is illustrated that the first and second electric supply contacts 121, 122 comprise a somewhat circular or at least semicircular shape and extend along the circumference of the drive unit housing 9 or cartridge holder 15. The complementary shaped counter supply contacts 221, 222 may vary with regard to their circumferential position as indicated by the dashed lines of the first and second counter supply contacts 221′, 222′. This allows for a rotation of the protective cap 14 relative to the inner or outer body 10, 11 within a predefined extend, e.g. as specified by fastener 124 and the mutually corresponding counter fastener 224 thereby maintaining an electrical contact.

As illustrated, the circumferential extent of the first and second electric counter supply contacts 221, 222 is slightly smaller than a circumferential gap between the first and the second electric supply contacts 121, 122. This way, a short circuit between the first and the second electric supply contact 121, 122 by any of the first or second counter supply contact 221, 222 can be effectively prevented.

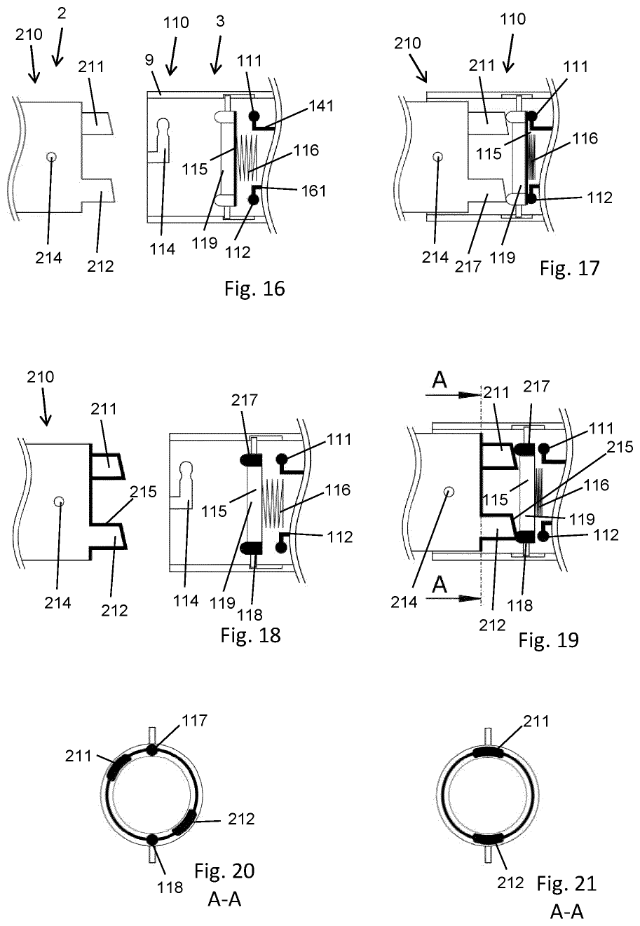

In the illustration of FIGS. 16-21 an example of a mechanical connection between the drive unit 3 and the container unit 2 is exemplary illustrated. The drive unit 3 comprises the first interface 110 complementary shaped and configured for engagement with a respective counter interface 210 as provided at the proximal end of the container unit 2. As illustrated, the first interface 110 comprises a receptacle configured and shaped to receive an insert section as provided at the proximal end of the container unit 2. The distal end of the drive unit 3, in particular a distal end of the drive unit housing 9 comprises a fastener 114 configured for engagement with a complementary shaped counter fastener 214 of the counter interface 210. As illustrated in FIGS. 16-19 the mutual connection of the fastener 114 and the counter fastener 214 may comprise or form a bayonet joint. Other types of connections, such as snap fit connections or screw-type connections are equally available or conceivable.

As further illustrated in FIG. 16 the counter interface 210 of the container unit 2 is provided with a first counter contact element 211 and with a second counter contact element 212. The first and the second counter contact elements 211, 212 comprise a somewhat beveled or slanted proximal end face. The first and second contact 111, 112 as provided on or in the first interface 110 may be selectively electrically connected by an electrical connector 115 movably disposed in or on the first interface 110.

There may be provided a carrier 119 longitudinally displaceable against or under the action of a contact spring 116. In the illustration of FIG. 16 the electrical connector 115 is provided on the carrier 119. It is out of contact from the first and the second electrical contact elements 111, 112. As the container unit 2 is connected to the drive unit 3 the counter contact elements 211, 212 will engage with the carrier 119 and urge the carrier 119 towards the proximal direction against the action of the contact spring 116. When reaching a final assembly configuration, in which the counter fastener 214 fully engages the fastener 114 the carrier 119 and the contact conductor 115 as provided on the carrier 119 have moved into a contact position with the first electrical contact element 111 and the second electric contact element 112. In this way an electrical contact between the first electric contact element 111 and the second electric contact element 112 is closed. There will be hence provided a closed conductor loop between the first electrical conductor path 141 and the second electrical conductor path 161.

With the further example of FIGS. 18 and 19 the electrical connector 115 comprises a first electrical contact element 117 and a second electrical contact element 118 located or arranged on the movable carrier 119. Here, the first and second contact elements 117, 118 of the electrical connector 115 are electrically isolated from each other. They may individually electrically contact with the first and the second electrical contacts 111, 112 of the first interface 110.

This way a mutual electrical contact between the first and the second electrical contacts 111, 112 is provided by a mechanical engagement with the first and the second counter contact elements 211, 212 that are provided with a separate contact conductor 215. Here, a surface of the first and second counter contact element 211, 212 may be provided with an electrically conducting structure or material. Moreover, the first and the second counter contact elements 211, 212 are electrically connected via the contact conductor 215. In a final assembly configuration, which is not yet reached in FIG. 19 the first electrical contact element 111 is electrically connected to the further contact element 117. The further contact element 117 is electrically connected to the counter contact element 211. The counter contact element 211 is permanently electrically connected to the counter contact element 212. Also, the counter contact element 212 is in electrical contact with the further contact element 118 of the electrical connector 115, which in turn is electrically connected to the second electric contact element 112.

In FIG. 20, a cross-section A-A is illustrated that represents the configuration before reaching a final assembly position and before the counter fastener 214 reaches an end position within the groove of the fastener 114. Here, the counter contact elements 211, 212 are located at a circumferential offset from the contact elements 117, 118. When reaching a final assembly position a respective contact configuration is obtained.

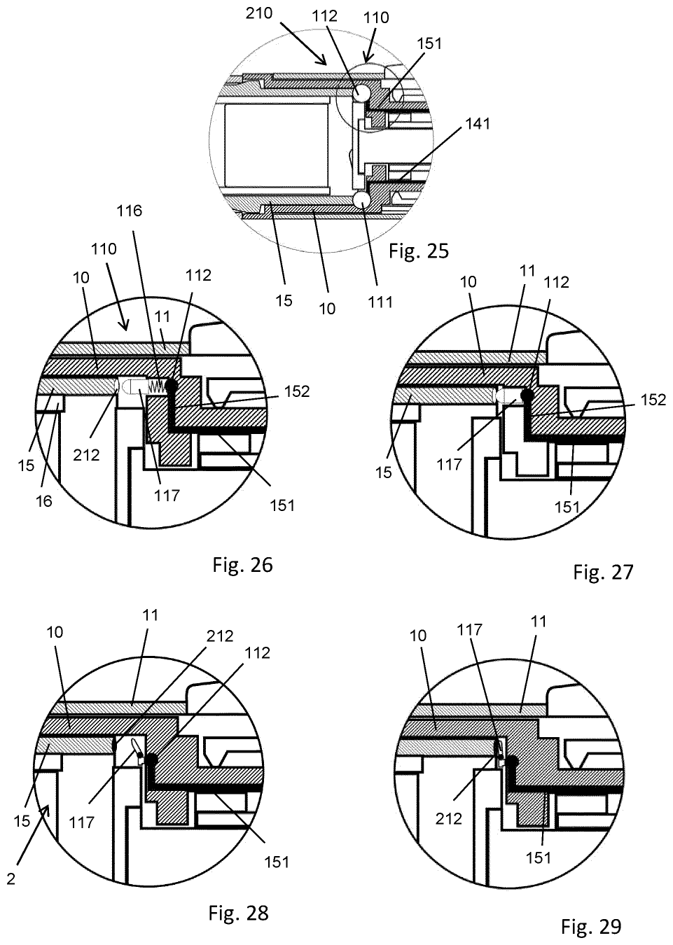

With the examples of FIGS. 25-29 another way of establishing an electrical contact between the first interface 110 and the counter interface 120 is schematically illustrated. Here, the first or second contacts 111, 112 are provided with an electrically conductive contact element 117, which is movably disposed or movably connected to the distal end 152 of the second electrical conductor path 151. The proximal end of the cartridge holder 15 may be provided with a respective counter contact element 212. The counter contact element 212 may be provided as an electrically conductive structure at a proximal end face of the sidewall of the cartridge holder 15. It may comprise an annular closed conductive ring structure on the proximal end or end face of the cartridge holder 15. In the configuration as illustrated in FIG. 26 the final assembly position has not yet been reached. Here, the counter contact element 212 and the contact element 117 are out of engagement.

When arriving in the final assembly configuration as shown in FIG. 27 the contact element 117 is in surface contact with the counter contact element 212. Moreover, the contact element 117 is mechanically biased by the contact spring 116. The contact spring 116 may be made of an electrically conductive material and may provide a respective transmission of electrical signals between the contact element 117 and the electrical conductor path 151.

With the further example as illustrated in FIGS. 28 and 29 the contact element 117 of the second electrical contact element 112 is implemented as a pliable or elastically deformable tongue, which is deformable or pivotable towards the proximal direction as it engages with the counter contact element 212 provided on the proximal end of the container unit 2. The counter contact element 212 may comprise an annular structure at the proximal end of the sidewall of the cartridge holder 15. In the proximal contact position it is electrically connected to both contact elements 111, 112. This way, the presence of a cartridge holder 15 and the correct mounting of the cartridge holder 15 to the drive unit 2 can be electronically detected.

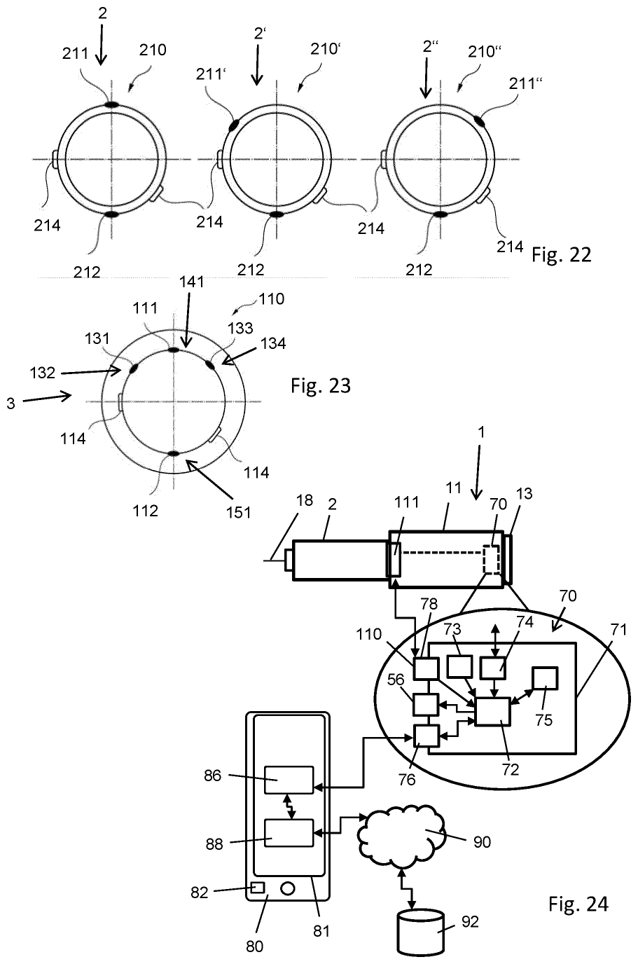

FIG. 22 is illustrative of a series of differently configured counter interfaces 210, 210′, 210″ of a series of different types of container units 2, 2′, 2″ which are typically equipped with a different drug container 16 and/or with a different medicament. The counter interfaces 210, 210′, 201″ are electrically encoded by the position or orientation of the counter contact elements 211, 212 as illustrated in FIG. 22. Moreover, the counter interfaces 210, 210′, 210″ are provided with an identical counter fastener 214 configured for mechanical engagement with a complementary shaped fastener 114 as the first interface 110 of the drive unit 3. In the present illustration there are provided two fastener 114 at a particular angular offset.

The counter fasteners 214 of the various container units 2, 2′, 2″ are provided at a respective angular offset. This way, the counter interfaces 210, 210′, 210″ can be only fastened in one discrete orientation with regard to the longitudinal axis as an axis A of rotation.

As illustrated in FIG. 23 the first interface 110 comprises not only a first electrical contact element 111 and a second electrical contact element 112 but also a third electrical contact element 131 and a fourth electrical contact element 133. The third contact element 131 is connected with a third longitudinal extending electrical conductor path 132. The fourth contact element 133 is connected with a respective for electrical conductor path 134. These supplemental conductor paths 133, 134 may be connected to the electronic unit 70 in the same or like a conducting manner as the first and second electrical conductor paths 141, 151.

With the numerous container units and counter interfaces 210, 210′, 210″ as shown in FIG. 22 there is always provided a common counter contact element 212 that is located in the same position relative to the counter fastener 214. With the numerous counter interfaces 210, 210′, 210″ the position of the further counter contact element 211, 211′, 211″ varies with regard to the circumferential direction relative to the counter contact element 212 and/or relative to the counter fasteners 214.

Only one of the contact elements 131, 111, 133 may be electrically connected to the counter contact element 211, 211′, 211″. For instance, with the counter interface 210, the counter contact element 211 is electrically connected with the counter contact element 212. With the counter interface 210′, the counter contact element 212 is electrically connected with the counter contact element 211′ and with the counter interface 210″ the counter contact element 212 is electrically connected with the counter contact element 211″.

Now and when the counter interface 210 is connected to the first interface 110 the electric contact element 112 gets in electrical contact with the counter contact element 212. Moreover, the contact element 111 gets in electrical contact with the counter contact element 211. Accordingly, the electronic unit 70 may sense or detect an electrical contact between the first and the second electric contact element 111, 112 and may thus identify the presence and/or correct assembly of the container unit 2 and the drive unit 3.

When the further counter interface 210′ is engaged with the first interface 110 the counter contact element 211′ gets in electrical contact with the third electric contact element 131. The counter contact element 212 gets in electrical contact with the second contact element 112. Accordingly, the electronic unit 70 may detect an electrical connection between the second and the third contact element 112, 131 and may thus identify the respective counter interface 210′.

Furthermore, and when the container unit 2 is equipped with the counter interface 210″ is assembled or connected with the first interface 110 there is provided an electrical contact between the second contact element 112 and the second counter contact element 212. In addition, there will be provided electrical contact between the counter contact element 211″ and the fourth contact element 133. Accordingly, and when the counter contact element 212 is electrically connected with the counter contact element 211″ there will be provided an electrical contact between the second contact element 112 and the fourth contact element 133. This electrical contact may be detected and sensed by the electronic circuit 70, which is accordingly able to distinguish between the differently electrically encoded container unit 2, 2′, 2″.

FIG. 24 shows a block diagram of the various injection devices 1 as described herein that are provided with an electronic unit 70. With the present example the electronic unit 70 is embedded inside the drug delivery device 1. It is arranged at or near the proximal end of the housing 6.

The electronic unit 70 comprises a printed circuit board 71 provided with a processor 72. The electronic unit 70 is further provided with a source of electric energy, typically implemented as an electric battery 73. The processor 72 and the battery 73 may be provided on opposite sides of the printed circuit board 71.

The electronic unit 70 further comprises an interface 78 electrically connected to at least one of the first and second electrical conductor paths 141, 161. With some examples, wherein the electronic unit 70 is movably disposed in the proximal housing component 14 or is connected to a dial extension movable relative to the proximal housing component the interface 78 comprises a sliding contact or is connected to a sliding contact 144, 154, 164, 174, 184, 194. This way the interface 78 and the electronic unit 70 remain in electrical contact with at least one of the interfaces 110, 120.

The electronic unit 70 may further comprise a communication interface, e.g. in form of a short range wireless communication interface 76, particularly configured to communicate with a corresponding short range communication interface 86 of an external electronic device 80.

The processor 72 is connected to the locking mechanism 56 and is operable to control operation of the locking mechanism 56.

Optionally, the electronic unit 70 is provided with a sensor 74 operable to detect or to measure movement of at least one movable component of the drive unit 3. By way of the sensor 74, which may be implemented as one of an optical sensor, a capacitive sensor, an inductive sensor, an optical sensor or as an acoustic sensor, operation of the drive unit 3 and hence operation of the injection device 1 can be supervised and monitored.

By way of the sensor 74 information regarding the size of the dose currently set, dialed or dispensed can be obtained and can be stored in a local storage 75. Data repeatedly captured or obtained during repeated and subsequent use of the injection device 10 can be stored in the storage 75. Such injection-related data stored in the storage 75 can be synchronized by the short range communication interface 76 with the external electronic device 80 and/or with an external database 92, e.g. hosted or provided by a healthcare provider.

Typically, the external electronic device 80 is implemented as a smart phone, as a smart watch or as a tablet computer. It may comprise a wide range communication interface 88 to establish a communication link to the external database 92 via a communication network 90. Typically, the external electronic device 80 is a mobile device or wearable device. It comprises a display 81 and/or a speaker 82 to communicate with a user.

The external electronic device 80 may be wirelessly paired to the electronic unit 70. Upon successful pairing a respective confirmation may be provided to a user, e.g. via the display 81 and/or via the speaker 82 of the external electronic device 80. Upon transferring of data or information between the electronic unit 70 and the external electronic device 80 a visual and/or audible confirmation may be provided to the user, e.g. by the electronic unit 70 and/or by the external electronic device 80.

The processor 72 may be configured to implement an automatic re-locking, e.g. after termination of each dispensing or in injecting procedure. Moreover, and based on signals obtainable from the sensor 74 the processor 72 may be provided with information being indicative of the remaining filling level of the medicament container 16. In this way and when the sensor 74 provides respective sensor signals that the medicament container 16 is substantially depleted or empty the processor 72 may invoke activation of the interlock 56 or may block an unlocking of the interlock 56. Removal of the medicament container 16 or insertion of the medicament container 16 as well as disconnection or reconnection of the container unit 2 may trigger an automated locking or disabling of the function of the drive unit 3 for setting and/or for dispensing of a dose.

As illustrated in FIG. 6 operation of the drug delivery devices as described herein may start at a time t1. Here, a protective cap 14 may be removed from the respective drug delivery device. Such a removal may be detected by the electronic control unit, e.g. when the electrical contact between the second interface 120 and the counter interface 220 is abrogated.

At a time t2 the electronic unit 70 may be configured to conduct a cartridge holder detection routine. Here, signals or an electrical contact provided by the first and/or second electric contact elements 111, 112 are evaluated by the electronic unit 70.

When a correct container unit 2 or cartridge holder 15 should be detected, the electronic unit 70 may switch into an activation mode. If a wrong cartridge holder 15 should be detected or if no cartridge holder should be detected the electronic unit 70 may be operable to generate an alert signal or may lock operation of the drive unit 3 by activating the interlock 56 as provided by the electromechanical actuator 50.

The electronic unit 70 in the dose button 13 may evaluate electric signal(s) from or induced by the cartridge holder 15 and may hence unlock the drive unit 3 if a correct or intended cartridge holder or container unit 2 should be present. The verification of a correct container unit is provided on the basis of the electrically detectable coding of the cartridge holder 15. In addition, there may be also conducted a user verification or an authentication procedure, e.g. by a wireless communication link with the external electronic device 80 at a time t3.

If all these requests are approved and if the locking mechanism 56 of the drive unit 3 as provided by the electromechanical actuator 50 should be locked the electronic unit 70 may generate an unlocking control signal and may transmit the respective unlocking or control signal via first and second electric control path 181, 191 thereby unlocking the drive unit 3.

As described above the control paths 181, 191 may coincide or may be electrically connected to the conductor paths 141, 151. Actuation of the electromechanical actuator 50 may then only require application of an increased voltage or current to activate or to switch the electromagnet 52. Thereafter, at a time t5 a user may set a dose of appropriate size and at a time t6 the user may conduct a respective injection procedure. At the end of an injection, hence at a time t7 the interlock of the drive unit 3 may be reactivated, e.g. by modifying the control signal from the electronic unit 70. At a time t8 re-attaching of the protective cap 14 may be detected.

In effect, the total number of conducting paths can be reduced by using only one common electric circuit for the cartridge holder detection signal and for the control signal for actuating the electromechanical actuator 50. As illustrated in FIG. 6 removing the protective cap 14 may wake up the device 1. Initially, a rather low voltage signal will be applied on the first and second electrical conductor paths 141, 151, e.g. for the purpose of cartridge holder detection and/or for the purpose of communication with the external electronic device 80. After verification of the cartridge holder 15 and/or after authentication of the user a high(er) voltage signal is applied to unlock the device.

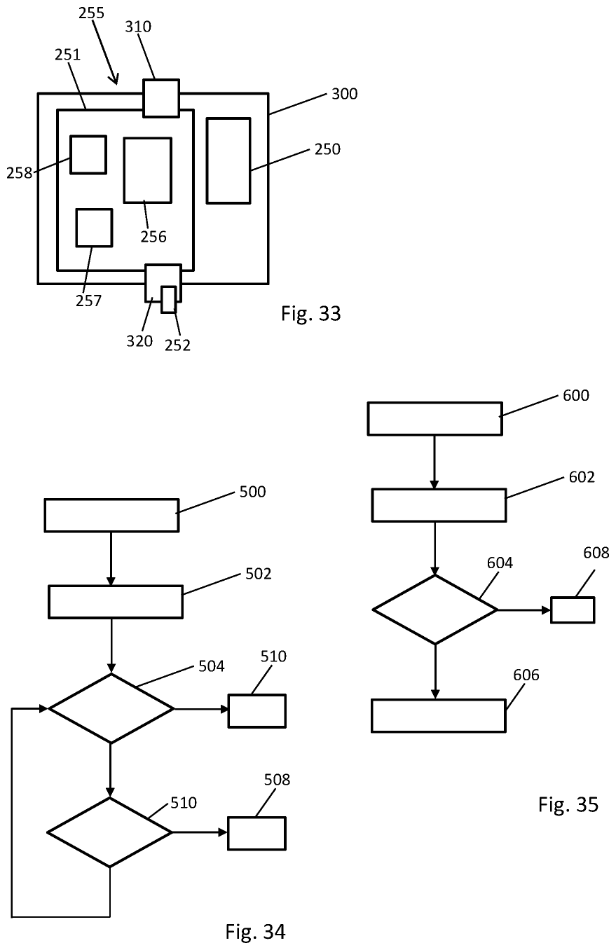

As further indicated in the block diagram of FIG. 33 the electrical power supply device 300 comprises a printed circuit board 251 with a logic circuit 255. The logic circuit 255 may comprise a processor 256, a timer 257, and optionally a communication interface 258. The communication interface 258 may be implemented as a wireless communication interface configured to wirelessly communicate with the external electronic device 80, which is illustrated in FIG. 24. Hence, the communication interface 258 may be implemented pursuant to commercially available wireless communication standards, such as Bluetooth, Wi-Fi, UWB or NFC.

The electrical power supply device 300 may be implemented as a kind of a power bank. The electrical power supply device 300 is detachably connectable to the body 10, 11 and/or to the housing 6 of the drug delivery device 1. The logic circuit 255 comprises a signal generator 254, which is operable to generate an out of time alert signal and/or to generate an energy depletion alert signal. The signal generator 254 is implemented and/or configured to generate at least one of a visual signal, an acoustic signal and/or a vibration or haptically perceivable signal.

The logic circuit 255 may be operable to detect an electrical connection between the supply interface 310 and the electric interface 130 of the drug delivery device 1. When the mutual electrical connection between the electrical interface 130 and the supply interface 310 is abrogated, e.g. due to a detachment of the protective cap 14 the processor 258 may be operable to start the timer 257. The processor 256 is further operable to stop the timer 257 in response to detect an electrical reconnection or reconnection of the supply interface 310 and the electrical interface 130. When the timer starts it start counting in discrete steps thereby increasing a count value per time interval.

If upon detection of a re-attachment of the electrical power supply device 300 to the drug delivery device 1 a count value of the timer previously started is below a predefined maximum count value the processor may be operable to stop the timer 257 and to reset the timer 257.

In situations, when a user does not reattach the protective cap 14 and hence the electrical power supply device 300 back onto the drug delivery device 1 within a predefined period of time, the counter 257 continues counting and the count value may exceed a predefined maximum count value. The processor 256 is then operable to cause the signal generator 254 to generate an out of time alert signal. Such an alert signal is generated when the count value of the timer exceeds the predefined maximum count value. This may occur, when the protective cap 14 is detached from the drug delivery device 1 over a comparatively long period of time. The out of time alert signal prompts the user to put the protective cap 14 back onto the drug delivery device 1.

Similarly, the processor 256 may be also operable to determine and/or to measure a charging level of the rechargeable energy reservoir 250. Here, the processor 256 is operable to cause the signal generator 254 to generate an energy depletion alert signal when the charging level drops below a predefined minimum charging threshold. The energy depletion alert signal is at least one of an acoustic signal, an optical or visual signal and a haptic signal.

Additionally or alternative to the generation of an alert signal directly perceivable by a user of the device, respective alerts or alert signals may be wirelessly transmitted via the communication interface 258 to the external electronic device 80. Then, a speaker, a display or a vibration module of the external electronic device may be operable to generate the user-perceivable alert signal.

The flowchart schematically illustrates one functional example of the electrical power supply device 300. In a first step 500 a user detaches the protective cap 14 from the drug delivery device 1. This detachment is detected by the logic circuit 255. Accordingly, and upon detecting the protective cap detachment the timer 257 is started or initiated in step 502. In the loop of steps 504, 510 it is checked if the protective cap is re-attached. If the cap re-attached in step 504, the method continues with step 510 in which the timer is stopped and is optionally subject to a reset.

If in step 504 reattachment of the cap is not detected it is checked in the proceeding step 506 the momentary count value of the timer is below the predefined threshold. If and as long as the timer value is below the threshold the method returns to step 504. If the timer is equal to or above the maximum count value and hence above the timer threshold there will be generated alert, i.e. the out of time alert in step 508.

In a further flowchart illustrating another function of the electrical power supply device 300 of FIG. 35 in a first step 600 the protective cap 14 and hence the electrical power supply device 300 is detached from the drug delivery device 1. In the subsequent step 602 the charging level of the rechargeable energy reservoir 250 is measured or determined. In step 604 the measured energy level is compared with a predefined minimum charging level. If the measured charging level is below or drops below the predefined minimum charging level the procedure continues with step 606. There, in step 606 an energy depletion alert signal is generated. Otherwise, and if in step 604 the energy level is above the predefined minimum charging threshold the method proceeds with step 608 and the electrical energy supply device may operate in a normal mode and may charge the battery 73 of the electronic unit 70 when re-attached to the drug delivery device 1 and when the supply interface 310 is appropriately electrically connected to the electrical interface 130 of the drug delivery device 1.

### REFERENCE NUMBERS

1 drug delivery device
2 container unit
3 drive unit
4 distal end
5 proximal end
6 housing
7 connection interface
8 connection interface
9 drive unit housing
10 inner body
11 outer body
12 window
13 dose button
14 protective cap
15 cartridge holder
16 drug container
17 stopper
18 injection needle
20 distal drive sleeve
21 proximal drive sleeve
22 drive sleeve coupler
23 distal clicker
24 proximal clicker
25 clutch spring
26 number sleeve
27 dial sleeve
28 clutch
29 plunger rod
30 last dose nut
40 device component
41 sidewall
42 outside surface
43 inside surface
44 longitudinal end face
45 longitudinal end face
46 threaded section
50 actuator
51 magnet
52 magnet
53 protrusion
54 recess
55 bending arm
56 locking mechanism
60 device component
61 sidewall
62 outside surface
63 inside surface
64 longitudinal end
65 longitudinal end
66 threaded section
70 electronic unit
71 printed circuit board
72 processor
73 battery
74 sensor
75 storage
76 communication interface
78 interface
80 external electronic device
81 display
82 speaker
86 communication interface
88 communication interface
90 network
92 database
110 interface
111 contact element
112 contact element
114 fastener
115 electrical connector
116 contact spring
117 contact element
118 contact element
119 carrier
120 interface
121 supply contact
122 supply contact
124 fastener
125 snap element
126 protrusion
127 recess
130 electrical interface
131 contact element
132 conductor path
133 contact element
134 conductor path
141 conductor path
142 distal end
143 proximal end
144 sliding contact
145 conductor path extension
146 contact conductor
151 conductor path
152 distal end
153 proximal end
154 sliding contact
155 conductor path extension
156 contact conductor
157 conductor branch
161 supply path
162 distal end
163 proximal end
164 sliding contact
165 supply path extension
166 contact conductor
171 supply path
172 distal end
173 proximal end
174 sliding contact
175 supply path extension
176 contact conductor
181 control path
182 distal end
183 proximal end
184 sliding contact
185 control path extension
191 control path
192 distal end
193 proximal end
194 sliding contact
195 control path extension
210 counter interface
211 counter contact element
212 counter contact element
214 counter fastener
215 contact conductor
220 counter interface
221 counter supply contact
222 counter supply contact
224 counter fastener
225 counter snap element
226 recess
227 protrusion
240 cap body
241 sidewall
242 distal end
243 proximal end
250 energy reservoir
251 printed circuit board
252 power connector
254 signal generator
255 logic circuit
256 processor
257 timer
258 communication interface
261 supply path
261 distal end
262 proximal end
271 supply path
271 distal end
272 proximal end
300 electrical power supply device
310 supply interface
320 charging interface
322 socket
324 inductive coupler
400 external power supply
421 cable
422 counter plug
424 inductive coupler

1.-19. (canceled)

20. An electrical power supply device for an electronic unit of a drug delivery device, the electrical power supply device comprising:
a body extending along a longitudinal direction and comprising a distal end and a proximal end;
a rechargeable energy reservoir arranged inside or on the body;
a supply interface electrically connected to the rechargeable energy reservoir,
wherein the supply interface is configured to electrically couple with an electrical interface of the drug delivery device, wherein the electrical interface is electrically connected to the electronic unit, and wherein the supply interface is configured to transfer electric energy from the rechargeable energy reservoir via the electrical interface to the electronic unit; and
a charging interface electrically connected to the rechargeable energy reservoir and configured to connect to an external power supply for charging the rechargeable energy reservoir.

21. The electrical power supply device according to claim 20, wherein the charging interface comprises a power connector.

22. The electrical power supply device according to claim 20, wherein the rechargeable energy reservoir is arranged inside the body,
wherein the supply interface is arranged at the proximal end or extends into the proximal end, and
wherein the charging interface is arranged at the distal end.

23. The electrical power supply device according to claim 20, wherein the electrical power supply device comprises a protective cap or is implemented as a protective cap of the drug delivery device implemented as a handheld injection device.

24. The electrical power supply device according to claim 20, wherein the body comprises a mechanical counter interface at the proximal end, wherein the mechanical counter interface is configured to detachably fasten the body to a complementary shaped mechanical interface of the drug delivery device.

25. The electrical power supply device according to claim 24, wherein the mechanical counter interface comprises a counter fastener to detachably engage with a complementary shaped fastener that is provided at the complementary shaped mechanical interface.

26. The electrical power supply device according to claim 25, wherein the supply interface comprises:
a first counter supply contact electrically connected to the rechargeable energy reservoir and electrically connectable to a complementary shaped first supply contact of the drug delivery device, and
a second counter supply contact electrically connected to the rechargeable energy reservoir and electrically connectable to a complementary shaped second supply contact of the drug delivery device.

27. The electrical power supply device according to claim 26, wherein the first counter supply contact and the second counter supply contact are arranged or integrated onto the mechanical counter interface.

28. The electrical power supply device according to claim 26, wherein the first counter supply contact and the second counter supply contact are integrated into the counter fastener.

29. The electrical power supply device according to claim 26, wherein the first counter supply contact and the second counter supply contact are arranged at the proximal end of the body.

30. The electrical power supply device according to claim 20, wherein the body comprises a sidewall confining a receptacle, which is open towards a proximal direction to receive at least a portion of the drug delivery device.

31. The electrical power supply device according to claim 30, wherein a mechanical counter interface is integrated into or is provided at a proximal end of the sidewall.

32. The electrical power supply device according to claim 31, wherein the supply interface comprises:
a first electric supply conductor path electrically connecting the supply interface with the rechargeable energy reservoir, and
a second electric supply conductor path isolated from the first electric supply conductor path and electrically connecting the supply interface with the rechargeable energy reservoir.

33. The electrical power supply device according to claim 32, wherein at least one of the first electric supply conductor path or the second electric supply conductor path is arranged on an inside surface or on an outside surface of the sidewall.

34. The electrical power supply device according to claim 20, further comprising a logic circuit, the logic circuit comprising a processor connected to the rechargeable energy reservoir and connected to at least one of the supply interface and the charging interface,
wherein the processor is configured to control at least one of:
charging the rechargeable energy reservoir via the charging interface, or
providing electrical energy to the drug delivery device via the supply interface.

35. The electrical power supply device according to claim 34, further comprising a timer and a signal generator controllable by the processor, wherein the processor is configured to:
start the timer in response to detecting an electrical disconnection of the supply interface from the electronic unit of drug delivery device,
stop the timer in response to detecting an electrical reconnection of the supply interface to the electronic unit of drug delivery device, and
cause the signal generator to generate an out of time alert signal when a count value of the timer exceeds a predefined maximum count value.

36. The electrical power supply device according to claim 35, wherein the processor is configured to determine a charging level of the rechargeable energy reservoir and wherein the processor is configured to cause the signal generator to generate an energy depletion alert signal when the charging level drops below a predefined minimum charging threshold.

37. A drug delivery device configured for setting and dispensing of a dose of a medicament, the drug delivery device comprising:
a housing;
an electronic unit arranged on or inside the housing; and
an electrical interface on or inside the housing electrically connected to the electronic unit and configured to electrically couple to a supply interface of an electrical power supply device,
wherein the electrical power supply device comprises:
a body extending along a longitudinal direction and comprising a distal end and a proximal end;
a rechargeable energy reservoir arranged inside or on the body;
the supply interface electrically connected to the rechargeable energy reservoir,
wherein the supply interface is configured to electrically couple with an electrical interface of the drug delivery device, wherein the electrical interface is electrically connected to the electronic unit, and wherein the supply interface is configured to transfer electric energy from the rechargeable energy reservoir via the electrical interface to the electronic unit; and
a charging interface electrically connected to the rechargeable energy reservoir and configured to connect to an external power supply for charging the rechargeable energy reservoir.

38. The drug delivery device according to claim 37, further comprising:
a mechanical interface; and
the electrical power supply device, wherein the electrical power supply device comprises a mechanical counter interface configured to detachably fasten the electrical power supply device to the mechanical interface.

39. The drug delivery device according to claim 37, wherein the rechargeable energy reservoir is arranged inside the body,
wherein the supply interface is arranged at the proximal end or extends into the proximal end, and
wherein the charging interface is arranged at the distal end.
